SEQUENCE LISTING (69 sequences)

SEQ ID NO: 1 - protein, 250 aa, Homo sapiens [sequence omitted]

SEQ ID NO: 2 - protein, 134 aa, Homo sapiens
VLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 3 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 4 - protein, 112 aa, Artificial Sequence
  anti-BCMA antibody VL sequence, antiBCMA Ab 1 VL
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKG

SEQ ID NO: 5 - protein, 117 aa, Artificial Sequence
  anti-BCMA antibody VH sequence, antiBCMA Ab 1 VH
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 6 - protein, 121 aa, Artificial Sequence
  anti-BCMA antibody VL sequence, antiBCMA Ab 2 VL
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 7 - protein, 108 aa, Artificial Sequence
  anti-BCMA antibody VH sequence, antiBCMA Ab 2 VH
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 8 - protein, 123 aa, Artificial Sequence
  anti-BCMA antibody VL sequence, antiBCMA Ab 3 VL
EVQLVESGGGLVKPGRSLRLSCTASGFTFGDYALSWFRQAPGKGLEWVGVSRSKAYGGTTDYAASVKGRFTISRDDSKSFAYLQMNSLKTEDTAVYYCCSSGYSSGWTPFDYWGQGTLVTVSS

SEQ ID NO: 9 - protein, 111 aa, Artificial Sequence
  anti-BCMA antibody VH sequence, antiBCMA Ab 3 VH
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTELTVLS

SEQ ID NO: 10 - protein, 27 aa, Artificial Sequence
  transmembrane domain
FWVLVVVGGVLACYSLLVTVAFIIFWV

SEQ ID NO: 11 - protein, 40 aa, Artificial Sequence
  CD28 co-stimulatory endodomain
SKRSRLLHSDYMNMTPRRPGPTRKHYQPYAPPRDFAAYRS

SEQ ID NO: 12 - protein, 36 aa, Artificial Sequence
  OX40 endodomain
RDQRLPPDAHKPPGGGSFRTPIQEEQADAHSTLAKI

SEQ ID NO: 13 - protein, 42 aa, Artificial Sequence
  4-1BB endodomain
KRGRKKLLYIFKQPFMRPVQTTQEEDGCSCRFPEEEEGGCEL

SEQ ID NO: 14 - protein, 112 aa, Artificial Sequence
  CD3zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 20 aa, Artificial Sequence
  signal peptide
METDTLLLWVLLLWVPGSTG

SEQ ID NO: 16 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 17 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 18 - protein, 20 aa, Artificial Sequence
  spacer sequence, human IgG1 hinge
AEPKSPDKTHTCPPCPKDPK

SEQ ID NO: 19 - protein, 237 aa, Artificial Sequence
  spacer sequence, IgG1 Hinge-Fc
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 20 - protein, 236 aa, Artificial Sequence
  spacer sequence, IgG1 Hinge - Fc modified to remove Fc receptor recognition motifs
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 21 - protein, 5 aa, Artificial Sequence
  Gly-Ser linker
SGGGS

SEQ ID NO: 22 - protein, 20 aa, Artificial Sequence
  cleavage site
YHADYYKQRLIHDVEMNPGP

SEQ ID NO: 23 - protein, 20 aa, Artificial Sequence
  cleavage site
HYAGYFADLLIHDIETNPGP

SEQ ID NO: 24 - protein, 20 aa, Artificial Sequence
  cleavage site
QCTNYALLKLAGDVESNPGP

SEQ ID NO: 25 - protein, 19 aa, Artificial Sequence
  cleavage site
ATNFSLLKQAGDVEENPGP

SEQ ID NO: 26 - protein, 19 aa, Artificial Sequence
  cleavage site
AARQMLLLLSGDVETNPGP

SEQ ID NO: 27 - protein, 20 aa, Artificial Sequence
  cleavage site
RAEGRGSLLTCGDVEENPGP

SEQ ID NO: 28 - protein, 20 aa, Artificial Sequence
  cleavage site
TRAEIEDELIRAGIESNPGP

SEQ ID NO: 29 - protein, 20 aa, Artificial Sequence
  cleavage site
TRAEIEDELIRADIESNPGP

SEQ ID NO: 30 - protein, 20 aa, Artificial Sequence
  cleavage site
AKFQIDKILISGDVELNPGP

SEQ ID NO: 31 - protein, 20 aa, Artificial Sequence
  cleavage site
SSIIRTKMLVSGDVEENPGP

SEQ ID NO: 32 - protein, 20 aa, Artificial Sequence
  cleavage site
CDAQRQKLLLSGDIEQNPGP

SEQ ID NO: 33 - protein, 20 aa, Artificial Sequence
  cleavage site
YPIDFGGFLVKADSEFNPGP

SEQ ID NO: 34 - protein, 402 aa, Artificial Sequence
  iCasp9 suicide gene sequence
MLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 35 - protein, 157 aa, Artificial Sequence
  RQR8 suicide gene sequence
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 36 - protein, 247 aa, Artificial Sequence
  TACI binder, 2H6 ScFv
EVQLQQSGPELVKPGASVRMSCKASGYTFTNYVMHWVKQKPGQGLEWIGYINPSNDDTKYTEKFKGKATLTSDKSSSTAYMELSSLTSEDSAVYYCARGTHGDYYALDYWGQGTSVTVSSGGGGAGGGGSGGGGSDIVLTQSPASLAVSLGQSVTISCRASESVEYYGTSLMQWYQQKPGQAPKLLIYGASNVESGVPARFSGSGSGTDFSLNIHPVEEDDIAMYFCQQSRKVPWTFGGGTKLEIKR

SEQ ID NO: 37 - protein, 5 aa, Artificial Sequence
  TACI binder, 2H6 CDR H1
NYVMH

SEQ ID NO: 38 - protein, 17 aa, Artificial Sequence
  TACI binder, 2H6 CDR H2
YINPSNDDTKYTEKFKG

SEQ ID NO: 39 - protein, 11 aa, Artificial Sequence
  TACI binder, 2H6 CDR H3
GTHGDYYALDY

SEQ ID NO: 40 - protein, 15 aa, Artificial Sequence
  TACI binder, 2H6 CDR L1
RASESVEYYGTSLMQ

SEQ ID NO: 41 - protein, 7 aa, Artificial Sequence
  TACI binder, 2H6 CDR L2
GASNVES

SEQ ID NO: 42 - protein, 7 aa, Artificial Sequence
  TACI binder, 2H6 CDR L3
QQSRKVP

SEQ ID NO: 43 - protein, 245 aa, Artificial Sequence
  TACI binder, 2G2 ScFv
QVTLKESGPGMLQPSQTLSLTCSFSGFSLSTFGMGVGWIRQPSGKGLEWLAHIWWDDAQYSNPALRSRLTISKDTSKNQVFLKIANVDTADTATYYCSRIHSYYSYDEGFAYWGQGTLVTVSSGGGGSGGGGSGGGGSDIVMTQSQKFMSTTVGDRVSITCKASQNVGTAVAWYQQKPGQSPKLLIYSASNRYTGVPDRFTGSGSGTDFTLTISNMQSEDLADYFCQQYSSYRTFGGGTKLEIKR

SEQ ID NO: 44 - protein, 7 aa, Artificial Sequence
  TACI binder, 2G2 CDR H1
TFGMGVG

SEQ ID NO: 45 - protein, 16 aa, Artificial Sequence
  TACI binder, 2G2 CDR H2
HIWWDDAQYSNPALRS

SEQ ID NO: 46 - protein, 14 aa, Artificial Sequence
  TACI binder, 2G2 CDR H3
RIHSYYSYDEGFAY

SEQ ID NO: 47 - protein, 11 aa, Artificial Sequence
  TACI binder, 2G2 CDR L1
KASQNVGTAVA

SEQ ID NO: 48 - protein, 7 aa, Artificial Sequence
  TACI binder, 2G2 CDR L2
SASNRYT

SEQ ID NO: 49 - protein, 6 aa, Artificial Sequence
  TACI binder, 2G2 CDR L3
QQYSSY

SEQ ID NO: 50 - protein, 6 aa, Artificial Sequence
  DXL motif, conserved 6-residue sequence
  MISC_FEATURE (1)..(1): Xaa may be Phe, Tyr or Trp
  misc_feature (3)..(3): Xaa can be any naturally occurring amino acid
  MISC_FEATURE (5)..(5): Xaa may be Val or Thr
  MISC_FEATURE (6)..(6): Xaa may be Arg or Gly
XDXLXX

SEQ ID NO: 51 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX

SEQ ID NO: 52 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 53 - protein, 7 aa, Artificial Sequence
  consensus Tobacco Etch Virus (TEV) cleavage site
ENLYFQS

SEQ ID NO: 54 - protein, 115 aa, Artificial Sequence
  TACI binder, 1G6 VH sequence
QVQLKQSGPGLVAPSQSLSITCTVSGFSLTSYGVDWVRQSPGKGLEWLGIIWGGGRTNYNSAFKSRLSISKDNSKSQVFLKMNSLQTDDTAMYYCASGDRAADYWGQGTSVTVSS

SEQ ID NO: 55 - protein, 5 aa, Artificial Sequence
  TACI binder, 1G6 CDR H1
SYGVD

SEQ ID NO: 56 - protein, 16 aa, Artificial Sequence
  TACI binder, 1G6 CDR H2
IIWGGGRTNYNSAFKS

SEQ ID NO: 57 - protein, 7 aa, Artificial Sequence
  TACI binder, 1G6 CDR H3
GDRAADY

SEQ ID NO: 58 - protein, 107 aa, Artificial Sequence
  TACI binder, 1G6 VL sequence
DIVMTQSQKFMSTTVGDRVTITCKASQNVGTAVAWYQQKPGQSPKLLIYSASNRYTGVPVRFTGSGSGTDFTLTINNMQSEDLADYFCQQYSSYPLTFGAGTKLELK

SEQ ID NO: 59 - protein, 11 aa, Artificial Sequence
  TACI binder, 1G6 CDR L1
KASQNVGTAVA

SEQ ID NO: 60 - protein, 7 aa, Artificial Sequence
  TACI binder, 1G6 CDR L2
SASNRYT

SEQ ID NO: 61 - protein, 7 aa, Artificial Sequence
  TACI binder, 1G6 CDR L3
QQYSSYP

SEQ ID NO: 62 - protein, 114 aa, Artificial Sequence
  TACI binder, 4B11 VH sequence
EVQLQQSVAELVRPGASVKLSCTASGFNIKNTYIHWVKQRPEQGLEWIGKIDPANGNSEYAPKFQGKATITADTSSNTAYLQLSSLTSEDTTIYYCTSGYGAYWGQGTTLTVSS

SEQ ID NO: 63 - protein, 5 aa, Artificial Sequence
  TACI binder, 4B11 CDR H1
NTYIH

SEQ ID NO: 64 - protein, 17 aa, Artificial Sequence
  TACI binder, 4B11 CDR H2
KIDPANGNSEYAPKFQG

SEQ ID NO: 65 - protein, 5 aa, Artificial Sequence
  TACI binder, 4B11 CDR H3
GYGAY

SEQ ID NO: 66 - protein, 111 aa, Artificial Sequence
  TACI binder, 4B11 VL sequence
DIVLSQSPSSLAVSIGEKVTLSCKSSQSLLYSSNQKNYLAWFQQKPGQSLKLLIYWASTREFGVPDRFTGSGSGTDFTLTISSVKTEDLAVYYCQQYYTWTFGGGTKLEIK

SEQ ID NO: 67 - protein, 17 aa, Artificial Sequence
  TACI binder, 4B11 CDR L1
KSSQSLLYSSNQKNYLA

SEQ ID NO: 68 - protein, 7 aa, Artificial Sequence
  TACI binder, 4B11 CDR L2
WASTREF

SEQ ID NO: 69 - protein, 6 aa, Artificial Sequence
  TACI binder, 4B11 CDR L3
QQYYTW

(19) United States
(12) Patent Application Publication
(10) Pub. No.: US 2019/0352409 A1
(43) Pub. Date: Nov. 21, 2019

(54) CHIMERIC ANTIGEN RECEPTOR

(71) Applicant: AUTOLUS LIMITED, London (GB)

(72) Inventors: Shimobi Onuoha, London (GB); Vijay Peddareddigari, London (GB); Lukas Stanczuk, London (GB); Mathieu Ferrari, London (GB); Biao Ma, London (GB)

(21) Appl. No.: 16/348,319

(22) PCT Filed: Nov. 10, 2017
(86) PCT No.: PCT/GB2017/053386

§ 371 Date: May 8, 2019

(30) Foreign Application Priority Data
Nov. 11, 2016 (GB) 1619097.7

(51) Int. Cl. C07K 16/28

(52) U.S. Cl. CPC C07K 16/2878; C07K 2317/31; C07K 2319/32; C07K 2319/03; C07K 2317/622

(57) ABSTRACT

The present invention provides a cell comprising first and second chimeric antigen receptors (CARs), which bind to different antigens, wherein the first CAR binds to Transmembrane activator and calcium modulator and cyclophilin ligand (CAML) interactor (TACI). The present invention also provides a cell comprising a tan CAR comprising first and second antigen-binding domains which bind to different antigens, wherein the first antigen binding domain binds the antigen Transmembrane activator and calcium modulator and cyclophilin ligand (CAML) interactor (TACI). The present invention further provides corresponding nucleic acid sequences and/or constructs, kits and vectors comprising said nucleic acid sequences and/or constructs, molecules and methods for making such cells. The cells may be used in cellular immunotherapy approaches for treating diseases such as multiple myeloma.

### FIELD OF THE INVENTION

The present invention relates to a cell comprising a chimeric antigen receptor (CAR) which binds antigen Transmembrane activator and calcium modulator and cyclophilin ligand (CAML) interactor (TACI). Such a cell is useful in the treatment of cancerous diseases, such as multiple myeloma.

### BACKGROUND TO THE INVENTION

Chimeric Antigen Receptors (CARs)

Chimeric antigen receptors are proteins which, in their usual format, graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1).

The most common form of these molecules use single-chain variable fragments (scFv) derived from monoclonal antibodies to recognize a target antigen. The scFv is fused via a spacer and a transmembrane domain to a signaling endodomain. Such molecules result in activation of the T-cell in response to recognition by the scFv of its target. When T cells express such a CAR, they recognize and kill target cells that express the target antigen.

Several CARs have been developed against tumour associated antigens, and adoptive transfer approaches using such CAR-expressing T cells are currently in clinical trial for the treatment of various cancers. For example, several clinical trials are in progress involving the use of CD19-targeted CAR T-cells for the treatment of haematological malignancies (Park et al 2016, Blood 127(26):3312-3320)

Cytokine Release Syndrome (CRS)

A significant drawback associated with CAR T-cell infusion, however, is the elicitation of toxicities, the most prevalent of which is cytokine release syndrome (CRS). In early CAR T-cell trials utilizing “first-generation” constructs (without costimulatory signaling elements), insufficient T-cell proliferation/cytokine production and lack of antitumor response were noted. The addition of costimulatory signaling in second-generation CAR design (e.g. CD28 or 41BB) translated to improved T-cell activation/expansion, cytokine production, and most notably dramatic antitumor responses in patients with hematologic malignancies. However, the “double-edged sword” of CAR T cells is demonstrated in the similarly impressive and potentially life-threatening CRS following CAR T-cell administration.

The hallmark of CRS is immune activation resulting in elevated inflammatory cytokines. Clinical and laboratory measures range from mild/moderate CRS (constitutional symptoms and/or grade-2 organ toxicity) to severe CRS (sCRS; grade≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been observed following CAR T-cell infusion.

The presence of CRS generally correlates with expansion and progressive immune activation of adoptively transferred cells. The cytokines which cause CRS come from the infused T-cells and the surrounding stromal cells, including monocytes and macrophages. A major cytokine implicated in CRS is IL-6 which is predominantly secreted by the myeloid cells (monocytes and macrophages). It can be neutralized and with it the signs and symptoms of CRS by using an anti-IL6 receptor antibody (e.g. toci) or anti-IL6 antibody (e.g.siltuximab).

It has been demonstrated that the degree of CRS severity is dictated by disease burden at the time of infusion as patients with high tumor burden experience a more sCRS. In reports of patients treated with CD19-specific CAR T cells for relapsed/refractory B-cell acute lymphoblastic leukemia, the incidence of sCRS has ranged from 19 to 43%, with variability likely due to differences in clinical identification of the syndrome, chimeric receptor designs, and infused cellular phenotypes. Clinical outcome is not predicated on the development of sCRS as patients may exhibit an antitumor response in the absence of this toxicity. However, in the context of hematologic malignancies the majority of patients who respond exhibit at least mild CRS (fever) following CAR T-cell infusion. Following diagnosis of CRS, a challenge has been choosing appropriate therapy to mitigate the physiological symptoms of uncontrolled inflammation without dampening the antitumor efficacy of the engineered cells. Systemic corticosteroid has been shown to rapidly reverse symptoms of sCRS without compromising initial antitumor response. However, prolonged use (e.g., >14 days) of high-dose corticosteroids has also resulted in ablation of the adoptively transferred CAR T-cell population potentially limiting their long-term antileukemia effect. As an effective alternative IL-6 receptor (IL-6R) blockade with the Food and Drug Administration-approved mAb, tocilizumab has demonstrated near-immediate reversal of CRS. However IL-6R blockade may have a negative effect on CAR T-cell proliferation, persistence, and most importantly, antitumor effect.

There is therefore a need for alternative mechanisms for avoiding and controlling CRS associated with CAR-T cell therapies.

Multiple Myeloma

Multiple Myeloma (myeloma) is a bone-marrow malignancy of plasma cells. Collections of abnormal plasma cells accumulate in the bone marrow, where they interfere with the production of normal blood cells. Myeloma is the second most common hematological malignancy in the U.S. (after non-Hodgkin lymphoma), and constitutes 13% of haematologic malignancies and 1% of all cancers. The disease is burdensome in terms of suffering as well as medical expenditure since it causes pathological fractures, susceptibility to infection, renal and then bone-marrow failure before death.

Unlike many lymphomas, myeloma is currently incurable. Standard chemotherapy agents used in lymphoma are largely ineffective for myeloma. Typically, these immunotherapeutic agents target a single antigen: for instance, Rituximab targets CD20; Myelotarg targets CD33; and Alemtuzumab targets CD52. However, since CD20 expression is lost in plasma cells, Rituximab cannot be used against myeloma. New agents such as Bortezamib and Lenolidomide are partially effective, but fail to lead to long-lasting remissions.

BCMA

BCMA is a transmembrane protein expressed in mature lymphocytes, e.g., memory B cells, plasmablasts, bone marrow plasma cells and myeloma cells. T cells transduced to express an anti-BCMA CAR have been shown to be capable of specifically killing myeloma cells from a plasmacytoma of a myeloma patient (Carpenter et al., 2013, Clin Cancer Res 19(8) 2048-60).

However, a consideration when targeting BCMA is the particularly low density of BCMA on myeloma cells, in comparison for instance with CD19 on a lymphoma cell (FIG. 2). Also, initial clinical data has suggested that treatment with T cells expressing an anti-BCMA CAR is associated with a loss of BCMA expression on myeloma cells over time and progression of BCMA-negative myeloma (Ali et al., (2016, Blood 128(13) 1688-1700). In addition, all patients which received the BCMA-CAR tested positive for CRS (cytokine release syndrome).

There is therefore a need for an improved therapeutic approach for the treatment of myeloma which addresses these issues.

### DESCRIPTION OF THE FIGURES

FIG. 1—Standard design of a Chimeric Antigen Receptor

The typical format of a chimeric antigen receptor is shown. These are type I transmembrane proteins. An ectodomain recognizes antigen. This is composed of an antibody derived single-chain variable fragment (scFv) which is attached to a spacer domain. This in turn is connected to a transmembrane domain which acts to anchor the molecule in the membrane. Finally, this is connected to an endodomain which acts to transmits intracellular signals to the cell. This is composed of one or more signalling domains.

FIG. 2—Expression data of BCMA on Myeloma

Myeloma cells from bone marrow samples from 39 multiple myeloma patients were isolated by a CD138+ magnetic bead selection. These cells were stained with the anti-BCMA monoclonal antibody J6MO conjugated with PE (GSK). Antigen copy number was quantified using PE Quantibrite beads (Becton Dickenson) as per the manufacturer's instructions. A box and whiskers plot of antigen copy number is presented along with the range, interquartile and median values plotted. We found the range is 348.7-4268.4 BCMA copies per cell with a mean of 1181 and a median of 1084.9.

FIG. 3—Ligand Specificity and Function Assignment of APRIL and BAFF

B-cell-activating factor (BAFF, TNFSF13B) interacts with BAFF-Receptor (BAFF-R, TNFRSF13C), B-cell membrane antigen (BCMA, TNFRSF17) and transmembrane activator and calcium modulator and cyclophilin ligand interactor (TACI, TNFRSF13B) while A proliferation-inducing ligand (APRIL, TNFSF13) interacts with only with BCMA, and TACI. BAFF-R activation affects peripheral B-cell survival, while BCMA may affect plasma cell survival.

FIG. 4—Surface Markers During Physiological Miceli Development

During development, B cells express different surface markers, some of which are targeted by therapeutic monoclonal antibodies as indicated. After initial development in the bone marrow, mature B cells migrate via the bloodstream to secondary lymphoid organs and may undergo germinal-centre reactions and class-switch recombination. They form memory B cells and antibody-secreting cells, which are short-lived plasmablasts or long-lived plasma cells. Fading bars indicate reports on the expression of surface markers are not uniform. Abbreviations: BAFF-R, B-cell-activating factor of the TNF family receptor (TNFRSF13C); BCMA, B-cell maturation antigen (TNFRSF17); TACI, transmembrane activator and CAML interactor (TNFRSF13B).

FIG. 5—Schematic diagram illustrating three possible TACI CAR designs.

In the first CAR (A), the human CD8 stalk domain is used as a spacer domain. In the second CAR (B), the hinge from IgG1 is used as a spacer domain. In the third CAR (C), the hinge, CH2 and CH3 domains of human IgG1 modified with the pva/a mutations described by Hombach et al (2010 Gene Ther. 17:1206-1213) to reduce Fc Receptor binding is used as a spacer. In all CARs, these spacers are connected to the CD28 transmembrane domain and then to a tripartite endodomain containing a fusion of the CD28, OX40 and the CD3-Zeta endodomain.

FIG. 6—Production of TACI-specific binders

a) ELISA screening to identify TACI positive antibody secreting hybridomas. 10 plates of parental hybridoma lines in total were screened and tested for TACI binding.

b) Confirmatory analysis of TACI binding by flow cytometry. Hybridoma lines were screened on TACI positive and BCMA positive SupT1 cells. The four hybridomas with the highest reactivity to SupT1 TACI cells were chosen for subcloning and characterisation

FIG. 7—TACI CARs

a) Schematic representation of TACI Chimeric antigen receptor cassette. TACI CAR2H6 was cloned into a third generation CAR format incorporating the endo-domains OX40 CD28 CD3 zeta.

b) 24 hour killing assay of TACI and BCMA expressing cells, comparing a CAR with a TACI antigen binding domain (2H6) and an equivalent CAR with a binding domain comprising truncated APRIL. Co-cultures were carried out at a 4:1 effector:target ratio c) Assay comparing IFNy release following co-culture 4:1 with TACI and BCMA expressing SupT1 cells

FIG. 8—Biacore affinity determination for TACI binding domains

FIG. 9—Schematic diagram illustrating the CARs described in Example 4

FIG. 10—Engagement of APRIL CAR-T cells with target cells results in production of high levels of IFNg and GM-CSF

Cytokine concentration in the media from a co-culture of APRIL CART cells and MM1s target cells. MM1s cells were seeded at 1:1 ratio either with APRIL CART cells or non-transduced (NT) control T cells. Following 48 h incubation, the media was collected and a panel of cytokines was measured by Luminex Multiplex assay (A) APRIL CAR-T cells; (B) Non-Transduced T cells.

FIG. 11—Combination of IFNg and GM-CSF induces production of IL-6 in monocytes

INFg in combination with GMC-SF induces IL-6 production in human primary monocytes and monocyte-like THP1 cells. (A) Human primary monocytes isolated from 3 healthy donors (n=3) or THP1 cells (n=1) were cultured for 48 h either alone or in the presence of IFNg (50 ng/ml) or GMC-SF (10 ng/ml) or both. Following the incubation, IL-6 levels were measured in the culture media by ELISA.

(B) Representative images of human primary monocytes showing distinct morphological changes in response to combination of INFg and GMC-SF following 48 h incubation.

FIG. 12—APRIL cells show cytotoxic activity against monocytes

Cytotoxic effect of APRIL CARTs on THP1 cells. (A) Summary of cytotoxicity assay results. APRIL CARTs, TRBC1 CARTs and non-transduced (NT) control cells were obtained from two matched donors (n=2). THP1 cells were co-cultured either with CART cells or their respective NT controls at 1:1 ratio for 72 h. THP1 lysis was analysed by flow cytometry. (B) Representative FACS plots from cytotoxicity assay with THP1 and CART cells.

FIG. 13—CAR mediated tumour cytolysis in the presence of autologous suppressor cells.

(a) M2 monocytes (M2), myeloid derived suppressor cells (MDSC) and dendritic cells (DC) TACI expression compared to unstimulated monocytes. (b) T cells either untransduced (blue), or transduced with APRIL-CAR (orange) or an anti-BCMA CAR having an scFv antigen binding domain based on an anti-BCMA monoclonal antibody (grey) were co-cultured at an E:T ratio of 1:4 with MM.1s cells in the presence of suppressor cells derived from autologous monocytes. M2 monocytes, MDSC and DCs were included at a ratio of 1:10 relative to MM1s cells.

### SUMMARY OF ASPECTS OF THE INVENTION

The present invention relates to CAR-based systems which target Transmembrane activator and calcium modulator and cyclophilin ligand interactor (TACI), together with another antigen. The CAR system may be:

a) A cell which co-expresses two or more different CARs, one of which binds TACI;

b) A tanCAR system with two or more antigen-binding domains, one of which binds TACI;

c) A composition comprising two or more populations of cells, each expressing a different CAR, one of which binds TACI; or

d) A method for treating a disease which comprises the administration of two or more populations of cells in separate administration steps, each population of cells expressing a different CAR, one of which binds TACI.

Thus, the first embodiment of the first aspect the present invention provides a cell comprising first and second chimeric antigen receptors (CARs) which bind to different antigens, wherein the first CAR binds to Transmembrane activator and calcium modulator and cyclophilin ligand interactor (TACI).

The second CAR may bind another antigen associated with multiple myeloma. For example, the second CAR may bind BCMA or BAFF.

The first CAR may comprise an immunoglobulin-like antigen binding domain. For example, the first CAR may comprise a TACI-binding scFv. The first CAR may lack the TACI-binding domain from a proliferation inducing ligand (APRIL).

The second embodiment of the first aspect of the invention provides a cell comprising a tanCAR comprising first and second antigen-binding domains which bind to different antigens, wherein the first antigen binding domain binds the antigen Transmembrane activator and calcium modulator and cyclophilin ligand interactor (TACT).

The second antigen-binding domain may bind another antigen associated with multiple myeloma. For example, the second antigen-binding domain may bind BCMA or BAFF.

The first antibody-binding domain may be an immunoglobulin-like antigen binding domain. For example, the first antibody-binding domain may comprise a TACI-binding scFv. The first antibody-binding domain may lack the TACI-binding domain from a proliferation inducing ligand (APRIL).

In a second aspect, the invention provides a nucleic acid sequence encoding a tanCAR as defined in the second embodiment of the first aspect of the invention.

The nucleic acid sequence may have the following structure:

AgB1-linker-AgB2-spacer-TM-endo

in which

AgB1 is a nucleic acid sequence encoding the first antigen-binding domain of the tanCAR;

linker is a nucleic acid sequence encoding a linker of the tanCAR;

AgB2 is a nucleic acid sequence encoding the second antigen binding domain of the tanCAR;

spacer is a nucleic acid sequence encoding a spacer of the tanCAR;

TM is a nucleic acid sequence encoding a transmembrane domain of the tanCAR;

endo is a nucleic acid sequence encoding an endodomain of the tanCAR;

which nucleic acid sequence, when expressed in a cell, encodes a polypeptide expressing the first and second antigen binding domains in tandem at the cell surface.

The linker may be or comprise a Gly-Ser flexible linker.

Alternative codons may be used in regions of sequence encoding the same or similar amino acid sequences, in order to avoid homologous recombination.

In a third aspect, the present invention provides a nucleic acid construct which encodes a first CAR and a second CAR as defined in the second embodiment of the first aspect of the invention.

The nucleic acid construct may have the following structure:

AgB1-spacer1-TM1-endo1-coexpr-AgB2-spacer2-TM2-endo2

in which

AgB1 is a nucleic acid sequence encoding the antigen-binding domain of the first CAR;

spacer1 is a nucleic acid sequence encoding a spacer of the first CAR;

TM1 is a nucleic acid sequence encoding a transmembrane domain of the first CAR;

endo1 is a nucleic acid sequence encoding an endodomain of the first CAR;

coexpr is a nucleic acid sequence enabling co-expression of the first and second CARs

AgB2 is a nucleic acid sequence encoding the antigen-binding domain of the second CAR;

spacer2 is a nucleic acid sequence encoding the spacer of the second CAR;

TM2 is a nucleic acid sequence encoding the transmembrane domain of the second CAR;

endo2 is a nucleic acid sequence encoding the endodomain of the second CAR;

which nucleic acid construct, when expressed in a cell, encodes a polypeptide which is cleaved at the cleavage site such that the first and second CARs are co-expressed at the cell surface.

The “coexpr” may encode a sequence comprising a self-cleaving peptide.

Alternative codons are used in regions of sequence encoding the same or similar amino acid sequences, in order to avoid homologous recombination.

In a fourth aspect there is provided a vector comprising a nucleic acid sequence according to the second aspect of the invention, or a nucleic acid construct according to the third aspect of the invention.

The vector may be a retroviral vector or a lentiviral vector or a transposon.

In a first embodiment of a fifth aspect of the invention, there is provided a kit which comprises

(i) a first nucleic acid sequence encoding a first CAR as defined in the first embodiment of the first aspect of the invention, which nucleic acid sequence has the following structure:

AgB1-spacer1-TM1-endo1

in which

AgB1 is a nucleic acid sequence encoding the antigen-binding domain of the first CAR;

spacer 1 is a nucleic acid sequence encoding the spacer of the first CAR;

TM1 is a nucleic acid sequence encoding the transmembrane domain of the first CAR;

endo 1 is a nucleic acid sequence encoding the endodomain of the first CAR; and

(ii) a second nucleic acid sequence encoding a second CAR as defined in claim 1 or 2, which nucleic acid sequence has the following structure:

AgB2-spacer2-TM2-endo2

in which

AgB2 is a nucleic acid sequence encoding the antigen-binding domain of the second CAR;

spacer 2 is a nucleic acid sequence encoding the spacer of the second CAR;

TM2 is a nucleic acid sequence encoding the transmembrane domain of the second CAR;

endo 2 is a nucleic acid sequence encoding the endodomain of the second CAR.

In a second embodiment of the fifth aspect of the invention kit comprising: a first vector which comprises the first nucleic acid sequence as defined in claim 15; and a second vector which comprises the second nucleic acid sequence as defined in claim 15.

The vectors may, for example, be integrating viral vectors, retroviral vectors, lentiviral vectors or transposons.

In a sixth aspect there is provided a method for making a cell according to the first aspect of the invention, which comprises the step of introducing: a nucleic acid sequence according to the second aspect of the invention; a nucleic acid construct according to the third aspect of the invention; a vector according to the fourth aspect of the invention or a kit of sequences or vectors according to the fifth aspect of the invention, into a cell.

The cell may be from a sample isolated from a subject.

In a first embodiment of a seventh aspect of the invention, there is provided a pharmaceutical composition comprising a plurality of cells according to the first aspect of the invention.

In a second embodiment of the seventh aspect of the invention, there is provided a pharmaceutical composition comprising:

(i) a first cell population expressing a first CAR; and

(ii) a second cell population expressing a second CAR

wherein the first and second CARs bind different antigens and the first CAR binds to Transmembrane activator and calcium modulator and cyclophilin ligand interactor (TACI).

In a first embodiment of an eighth aspect, the present invention provides a method for treating and/or preventing a disease, which comprises the step of administering a pharmaceutical composition according to the seventh aspect of the invention to a subject.

In a second embodiment of an eighth aspect, the present invention provides a method for treating and/or preventing a disease, which comprises the following steps:

(i) administration of a first cell population which expresses a first CAR

(ii) administration of a second cell population which expresses a second CAR

wherein the first and second CARs bind different antigens and wherein the first CAR binds to Transmembrane activator and calcium modulator and cyclophilin ligand interactor (TACT).

The method may comprise the following steps:

(i) isolation of a cell-containing sample from a subject;

(ii) transduction or transfection of the cells with: a nucleic acid sequence according to the second aspect of the invention; a nucleic acid construct according to the third aspect of the invention; a vector according to the fourth aspect of the invention or a kit of sequences or vectors according to the fifth aspect of the invention; and

(iii) administering the cells from (ii) to the subject.

In a ninth aspect, there is provided a kit for use in a method according to the eighth aspect of the invention, which comprises:

(i) a first cell population which expresses a first CAR

(ii) a second cell population which expresses a second CAR wherein the first and second CARs bind different antigens and wherein the first CAR binds to Transmembrane activator and calcium modulator and cyclophilin ligand interactor (TACI).

In a tenth aspect, there is provided a pharmaceutical composition according to the seventh aspect of the invention for use in treating and/or preventing a disease.

In an eleventh aspect, there is provided the use of a cell according to the first aspect of the invention in the manufacture of a medicament for treating and/or preventing a disease.

In connection with the eighth, tenth and eleven aspects of the invention, the disease may be a cancer, such as a mature B cell malignancy. The disease may, for example, be multiple myeloma.

In a twelfth aspect there is provided a tanCAR comprising a first and second antigen-binding domain which bind to different antigens, wherein the first antigen binding domain binds the antigen Transmembrane activator and calcium modulator and cyclophilin ligand (CAML) interactor (TACI).

In a thirteenth aspect there is provided a method for avoiding and/or reducing cytokine release syndrome in a subject which comprises the step of administering an anti-TACI agent to the subject.

The anti-TACI agent may be or comprise an anti-TACI antibody. The agent may be, for example, an antibody-drug conjugate (ADC), a bispecific antibody or a bispecific T-cell engager (BiTE).

The anti-TACI agent may be or comprise a cell which expresses and anti-TACI chimeric antigen receptor (CAR).

The anti-TACI CAR may comprise two separate components: an antigen-binding component, which comprises an antigen-binding domain and a transmembrane domain; and an intracellular signalling component which comprises an endodomain. The antigen binding component and intracellular signalling component associate at the cell surface to form a functional CAR complex.

The antigen binding component and intracellular signalling component may associate only in the presence or absence of certain molecule, as described in WO2015/150771 and WO2016/030691 respectively. The molecule, for example tetracycline, may cause dissociation of the antigen binding component and intracellular signalling component, reducing or stopping CAR function.

The anti-TACI CAR may have an antigen binding domain which comprises the BCMA binding domain from APRIL, as described in WO2015/052538. The antigen binding domain may comprise the sequence shown above as SEQ ID No. 2 or a fragment or variant thereof which retains TACI binding capacity.

The anti-TACI agent may be or comprise a cell according to the first aspect of the invention.

The anti-TACI agent may inhibit cytokine secretion by myeloid cells in the subject. The anti-TACI agent may kill myeloid cells in the subject, for example by apoptosis.

There is also provided an anti-TACI agent as defined above for use in a method for avoiding and/or reducing cytokine release syndrome in a subject.

There is also provided the use of anti-TACI agent as defined above in the manufacture of a medicament for use in avoiding and/or reducing cytokine release syndrome in a subject.

The sole targeting of a single antigen can result in tumour escape by modulation of the antigen due to the high mutation rate inherent in cancers. Targeting BCMA alone with a CAR approach has been shown to result in relapse with BCMA-negative myeloma (Ali et al 2016). The co-targeting of TACI, another myeloma associated antigen, should avoid such escape.

The co-targetting of two antigens also address the problem of low BCMA expression at the surface of multiple myeloma cells: when TACI is targeted in addition to another antigen, the effective overall antigen concentration on the target cell recognisable by the CAR system is increased.

BCMA is expressed by germinal centre B cells, while TACI is expressed predominantly by splenic transitional type 2 and marginal zone B cells, as well as activated B cells, (see FIG. 5). Targeting BCMA alone with a CAR T cell approach is likely to spare myeloma precursor cells. In addition, targeting BCMA alone can result in an increase in APRIL and BAFF secretion by stromal cells thereby promoting survival and growth of myeloma precursor cells. For these two reasons, targeting BCMA alone is likely to result in poor durability, lasting only as long as CAR-T cell persistence.

Targeting TACI, on the other hand, should result in better clearance of plasma blast and late pre-b cells, leading to better durability of response.

TACI is expressed on monocytes and macrophages which secrete cytokines such as IL-6, which can cause CRS.

Pre-treatment, co-treatment or post-treatment with an anti-TACI agent can therefore be used to avoid and/or reduce CRS in a CAR T cell immunotherapy. This approach is better than treatment of the patient with an ant-IL6 or anti-IL6 receptor antibody because it is longer lasting and will decrease, not only IL6, but other CRS-associated cytokines produced by monocytes and macrophages.

### DETAILED DESCRIPTION

Chimeric Antigen Receptors (CARS)

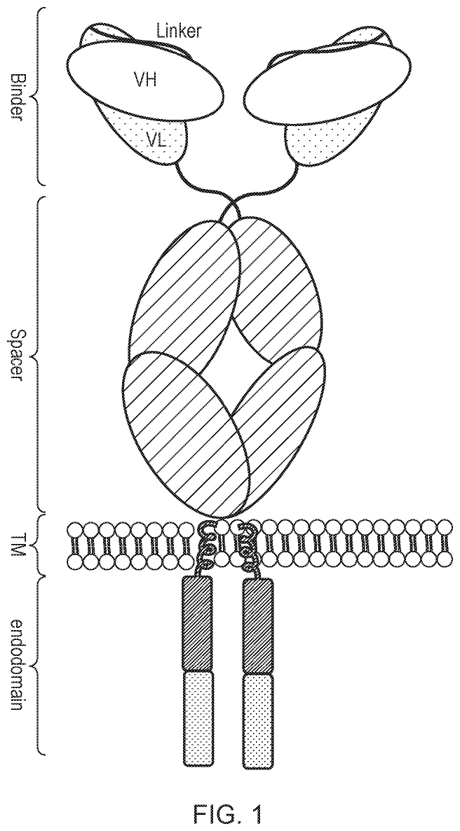

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcϵR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, many cancer-specific T cells can be generated for adoptive cell transfer. When the CAR binds the target-antigen, this results in the transmission of an activating signal to the T-cell it is expressed on. Thus, the CAR directs the specificity and cytotoxicity of the T cell towards tumour cells expressing the targeted antigen.

OR Gate

In a first embodiment, the first aspect of the invention relates to a cell which co-expresses a first CAR and a second CAR, wherein one CAR binds antigen TACI and the other CAR bind a different antigen, such that a the cell can recognize a target cells expressing either TACI and/or the other antigen as markers.

The second CAR may, for example, bind an antigen characteristic of a multiple myeloma cell, such as BCMA or BAFF.

The cell therefore comprises an OR gate. Logic gates for chimeric antigen receptor systems recognising a pattern of antigens are described in WO2015/075468.

The first and second CAR may be produced as a polypeptide comprising both CARs, separated by a cleavage site. Cleavage at the cleavage enables the two CARs to be expressed as separate entities at the cell surface.

Tandem CARs (TanCARs)

Bispecific CARs known as tandem CARs or TanCARs have been developed to target two or more cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position. When a TanCAR binds either or both of the target antigens, this results in the transmission of an activating signal to the cell it is expressed on.

Grada et al (2013, Mol Ther Nucleic Acids 2:e105) describes a TanCAR which includes a CD19-specific scFv, followed by a Gly-Ser linker and then a HER2-specific scFv. The HER2-scFv was in the juxta-membrane position, and the CD19-scFv in the distal position. The TanCAR was shown to induce distinct T cell reactivity against each of the two tumour restricted antigens. This arrangement was chosen because the respective lengths of HER2 (632 aa/125A) and CD19 (280aa, 65A) lends itself to that spatial arrangement. It was also known that the HER2 scFv bound the distalmost 4 loops of HER2.

The second embodiment of the first aspect of the invention relates to a cell which comprises a TanCAR comprising two antigen binding specificities in tandem where the first antigen binding domain binds to the antigen TACI and the second antigen binding domain binds to a different antigen.

In the TanCAR of the present invention, the TACI-binding antigen binding domain may be juxtaposed to the membrane and the other antigen-binding domain may be in a distal position, or vice versa.

The second antigen binding domain may bind to another antigen expressed on myeloma cells, such as BCMA or BAFF-R.

Antigen Binding Domain

The antigen binding domain is the portion of CAR or TanCAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen-binding domain may comprise an immunoglobulin-like antigen binding domain, such as an scFv.

TACI

Transmembrane activator and calcium modulator and cyclophilin ligand (CAML) interactor) TACI (UniProtKB: O14836) is a regulator in immune responses, and like BCMA, is preferentially expressed in mature lymphocytes such as CD27+ memory B cells, especially marginal zone B cells, bone marrow plasma cells and myeloma cells. Additionally, TACI is expressed on macrophages and mediates macrophage survival. TACI is a lymphocyte-specific member of the tumour necrosis factor (TNF) receptor superfamily, also known as Tumour necrosis factor receptor superfamily member 13B (TNFRSF13B), and can be shed from cells' surface and circulate in its soluble form. In contrast with BCMA, TACI is notably absent from germinal center B cells. TACI is known to function as the receptor for TNFSF13/APRIL and TNFSF13B/BAFF and binds both ligands with high affinity. TACI inhibits B cell expansion and promotes the differentiation and survival of plasma cells.

TACI is a member of the tumor necrosis factor receptor (TNFR) superfamily and serves as a key regulator of B cell function. TACI binds two ligands, APRIL and BAFF, with high affinity and contains two cysteine-rich domains (CRDs) in its extracellular region. However, another form of TACI exists in which the N-terminal CRD is removed by alternative splicing. It has been shown that this shorter form is capable of ligand-induced cell signaling and that the second CRD alone (TACI_d2) contains full affinity for both ligands (Hymowitz et al 2005 Am Soc. Biochem. And Mol. Biol. Inc 280(8) 7218-7227).

The crystal structure of TACI_d2 has been solved along with cocrystal structures of APRILTACI_d2 and APRIL BCMA complexes (Hymowitz et al 2005, as above).

The CRDs of TACI, together with CRDs of other TNFRs, have a common sequence feature, the so-called DXL motif, which consists of a conserved 6-residue sequence (Phe/Tyr/Trp)-Asp-Xaa-Leu-(Val/Thr)-(Arg/Gly) (SEQ ID No. 50). This motif is required for binding to either APRIL or BAFF. The receptor motif binds in a hydrophobic pocket and interacts with two conserved arginine residues on the BAFF surface.

The antigen binding domain of CAR or tanCAR which binds to TACI may be any domain which is capable of binding TACI. For example, the antigen binding domain may comprise a TACI binder derivable from one of the commercially available anti-TACI antibodies listed in the following table:

| Anti-TACI Ab | Company |
|---|---|
| 1A1 | BioLegend |
| ab5994 | Abcam |
| Ab79023 | Abcam |
| 11H3 | Affymetrix eBioscience |

The antigen binding domain may comprise one of the TACI binders described in Example 2, i.e. from clones 2H6, 2G2, 1G6 or 4B11.

These TACI binders have the following sequences:

2H6
ScFv:
(SEQ ID No. 36)
EVQLQQSGPELVKPGASVRMSCKASGYTFTNYVMHWVKQKPGQGLEWIGY
INPSNDDTKYTEKFKGKATLTSDKSSSTAYMELSSLTSEDSAVYYCARGT
HGDYYALDYWGQGTSVTVSSGGGGAGGGGSGGGGSDIVLTQSPASLAVSL
GQSVTISCRASESVEYYGTSLMQWYQQKPGQAPKWYGASNVESGVPARFS
GSGSGTDFSLNIHPVEEDDIAMYFCQQSRKVPWTFGGGTKLEIKR
CDR H1:
(SEQ ID No. 37)
NYVMH
CDR H2:
(SEQ ID No. 38)
YINPSNDDTKYTEKFKG
CDR H3:
(SEQ ID No. 39)
GTHGDYYALDY
CDR L1:
(SEQ ID No. 40)
RASESVEYYGTSLMQ
CDR L2:
(SEQ ID No. 41)
GASNVES
CDR L3:
(SEQ ID No. 42)
QQSRKVP
2G2
ScFv:
(SEQ ID No. 43)
QVTLKESGPGMLQPSQTLSLTCSFSGFSLSTFGMGVGWIRQPSGKGLEWL
AHIWWDDAQYSNPALRSRLTISKDTSKNQVFLKIANVDTADTATYYCSRI
HSYYSYDEGFAYWGQGTLVTVSSGGGGSGGGGSGGGGSDIVMTQSQKFMS
TTVGDRVSITCKASQNVGTAVAWYQQKPGQSPKLLIYSASNRYTGVPDRF
TGSGSGTDFTLTISNMQSEDLADYFCQQYSSYRTFGGGTKLEIKR
CDR H1:
(SEQ ID No. 44)
TFGMGVG
CDR H2:
(SEQ ID No. 45)
HIWWDDAQYSNPALRS
CDR H3:
(SEQ ID No. 46)
RIHSYYSYDEGFAY
CDR L1:
(SEQ ID No. 47)
KASQNVGTAVA
CDR L2:
(SEQ ID No. 48)
SASNRYT
CDR L3:
(SEQ ID No. 49)
QQYSSY
1G6
VH:
(SEQ ID No. 54)
QVQLKQSGPGLVAPSQSLSITCTVSGFSLTSYGVDWVRQSPGKGLEWLGI
IWGGGRTNYNSAFKSRLSISKDNSKSQVFLKMNSLQTDDTAMYYCASGDR
AADYWGQGTSVTVSS
CDR H1:
(SEQ ID No. 55)
SYGVD
CDR H2:
(SEQ ID No. 56)
IIWGGGRTNYNSAFKS
CDR H3:
(SEQ ID No. 57)
GDRAADY
VL:
(SEQ ID No. 58)
DIVMTQSQKFMSTTVGDRVTITCKASQNVGTAVAWYQQKPGQSPKLLIYS
ASNRYTGVPVRFTGSGSGTDFTLTINNMQSEDLADYFCQQYSSYPLTFGA
GTKLELK
CDR L1:
(SEQ ID No. 59)
KASQNVGTAVA
CDR L2:
(SEQ ID No. 60)
SASNRYT
CDR L3:
(SEQ ID No. 61)
QQYSSYP
4B11
VH:
(SEQ ID No. 62)
EVQLQQSVAELVRPGASVKLSCTASGFNIKNTYIHWVKQRPEQGLEWIGK
IDPANGNSEYAPKFQGKATITADTSSNTAYLQLSSLTSEDTTIYYCTSGY
GAYWGQGTTLTVSS
CDR H1:
(SEQ ID No. 63)
NTYIH
CDR H2:
(SEQ ID No. 64)
KIDPANGNSEYAPKFQG
CDR H3:
(SEQ ID No. 65)
GYGAY
VL:
(SEQ ID No. 66)
DIVLSQSPSSLAVSIGEKVTLSCKSSQSLLYSSNQKNYLAWFQQKPGQSL
KLLIYWASTREFGVPDRFTGSGSGTDFTLTISSVKTEDLAVYYCQQYYTW
TFGGGTKLEIK
CDR L1:
(SEQ ID No. 67)
KSSQSLLYSSNQKNYLA
CDR L2:
(SEQ ID No. 68)
WASTREF
CDR L3:
(SEQ ID No. 69)
QQYYTW

The present invention provides an anti-TACI agent, such as an antibody, scFv, CAR or tanCAR which comprises a TCI-binding domain based on one of the clones 2H6, 2G2, 1G6 or 4B11. The anti-TACI agent may comprise one or more CDRs having the sequences shown as SEQ ID Nos 37-42; 44-49; 55-57; 59-61; 63-65; 67-69. The agent may comprise one of the following groups of six CDRs:

SEQ ID Nos 37-42; SEQ ID Nos 44-49; SEQ ID Nos 55-57 and 59-61; or SEQ ID Nos 63-65 and 67-69.

The anti-TACI agent may comprise the VH and/or VL sequences shown in SEQ ID No. 36 or 43 (VH is before the serine-glycine linker, VL is after the serine-glycine linker). The anti-TACI agent may comprise the VH sequence shown in SEQ ID No. 54 or 62 and/or the VL sequence shown in SEQ ID No. 58 or 66. The anti-TACI agent may comprise the scFv shown in SEQ ID 36 or 43 or an scFv formed by !inkling SEQ ID No. 54 with SEQ ID No. 58 or SEQ ID No. 62 with SEQ ID No. 66.

Wth all of the above-mentioned sequences, the anti-TACI agent may comprise a sequence having 80%, 85%, 90%, 95% or 98% identity to the given sequence, provided that the variant sequence retains the ability to bind TACI.

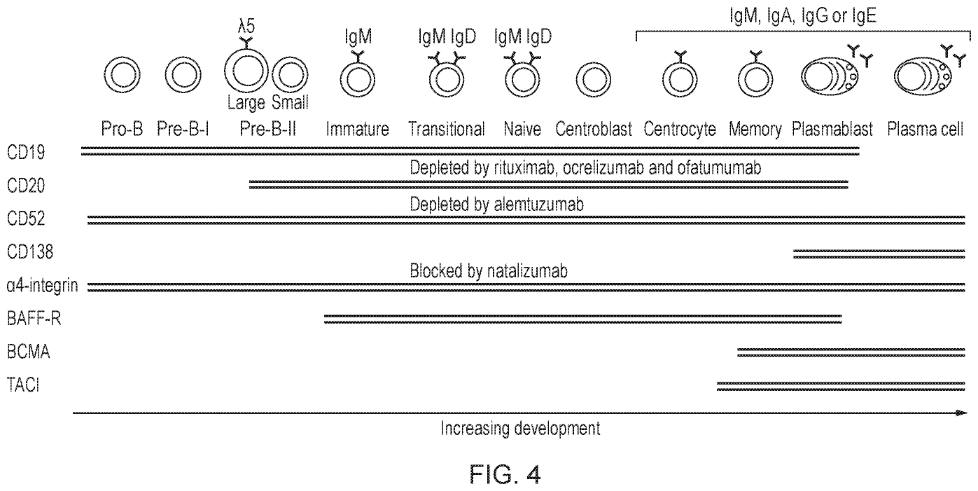

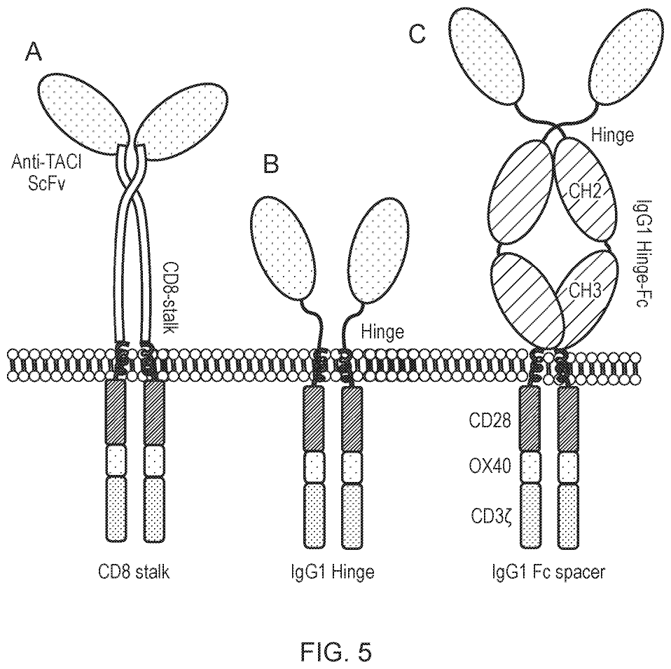

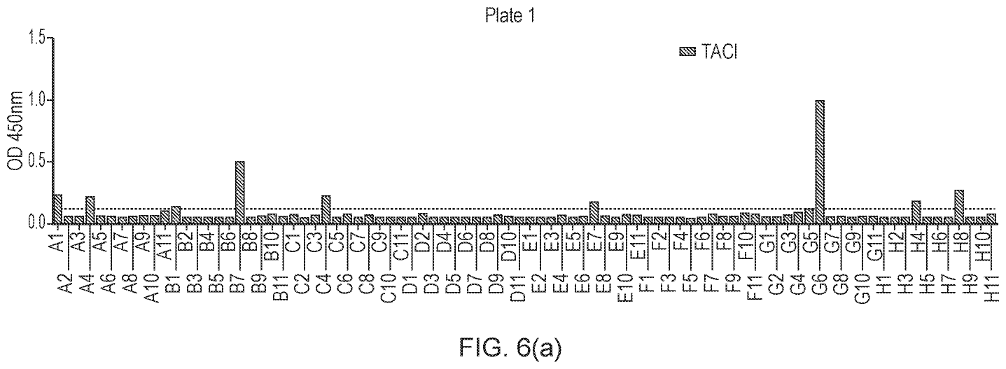

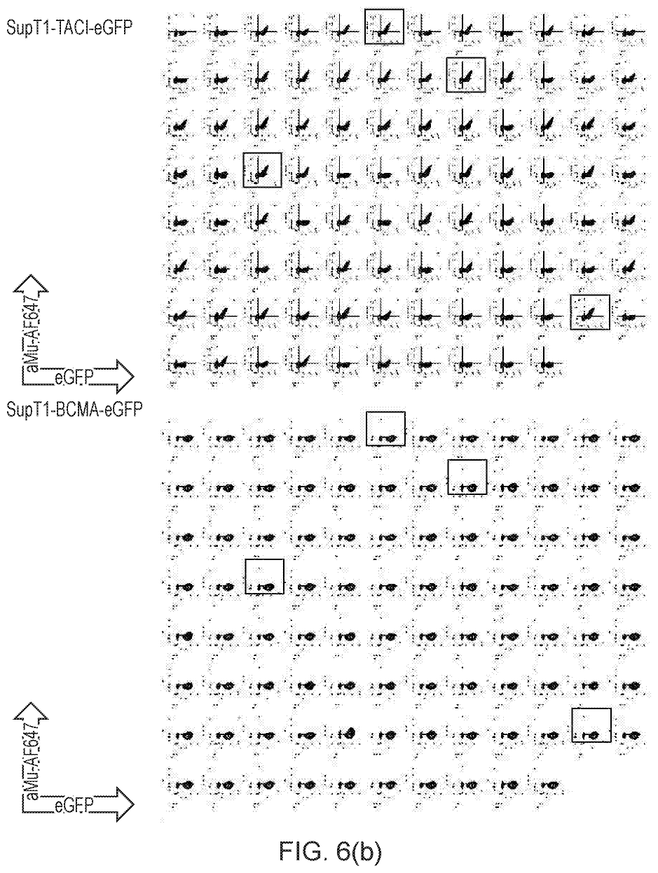

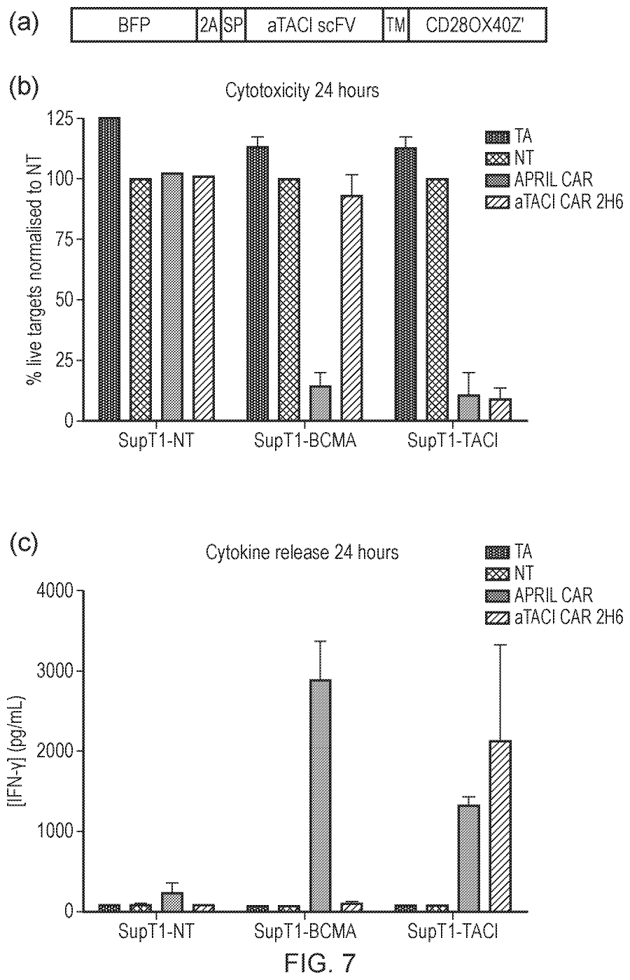

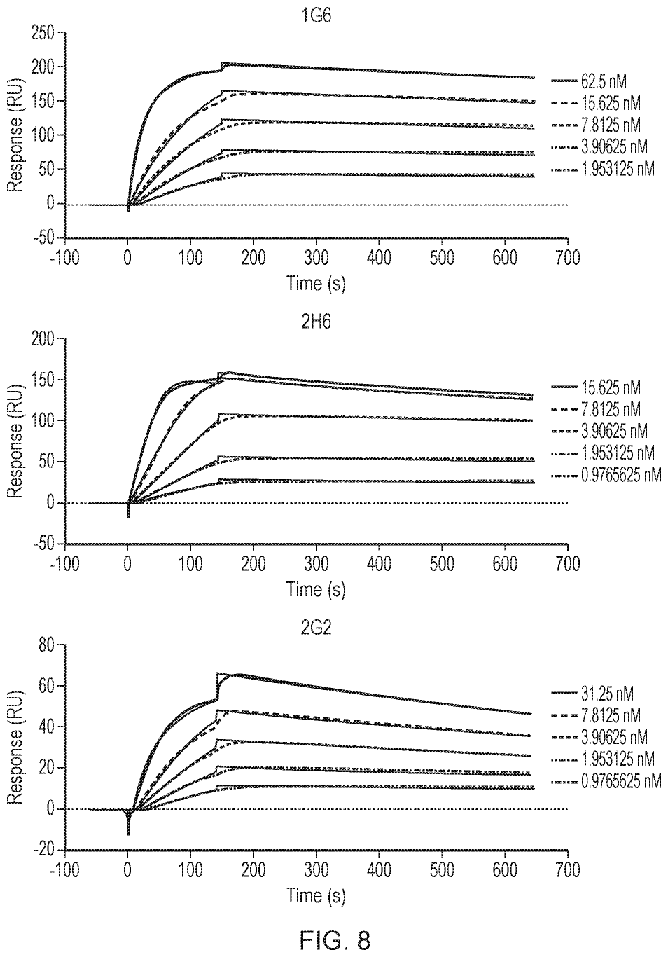

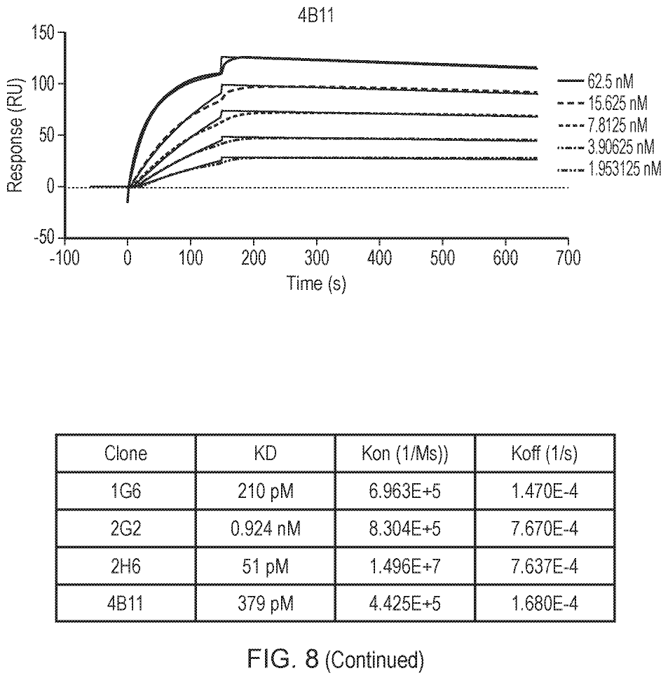

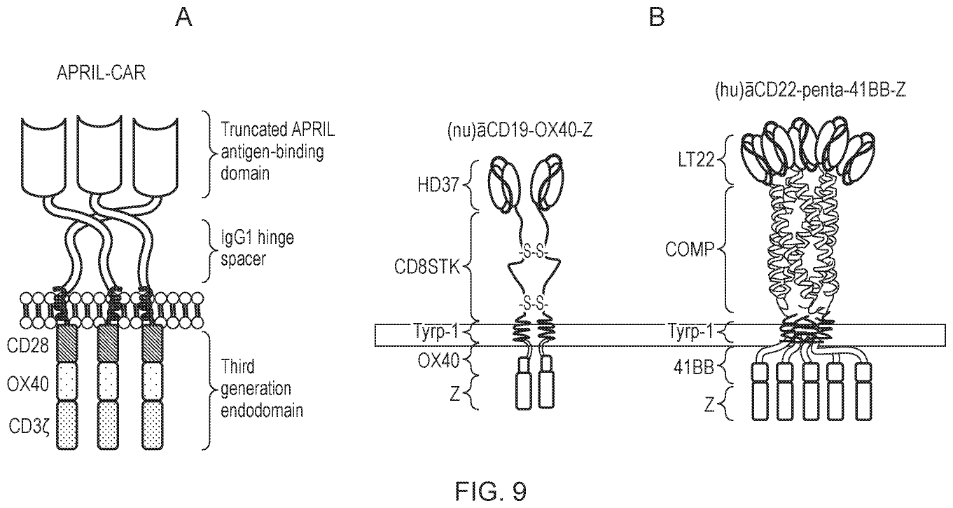

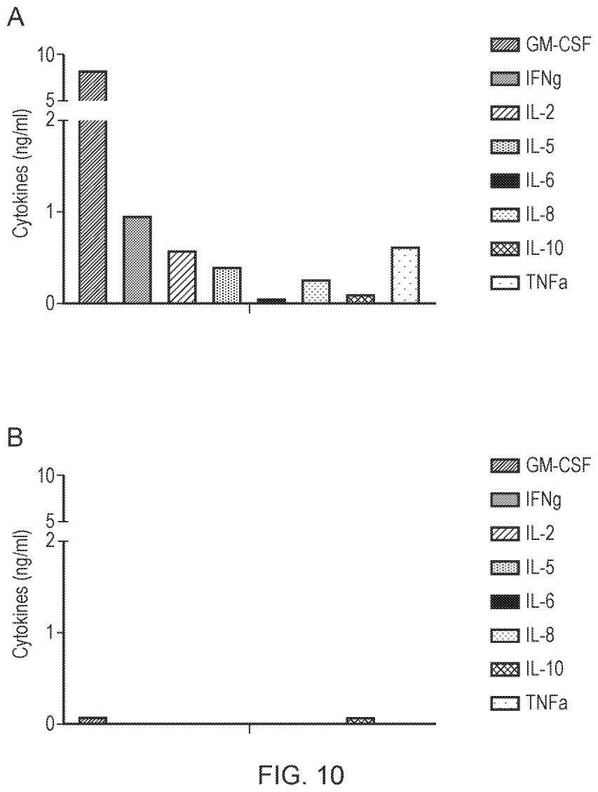

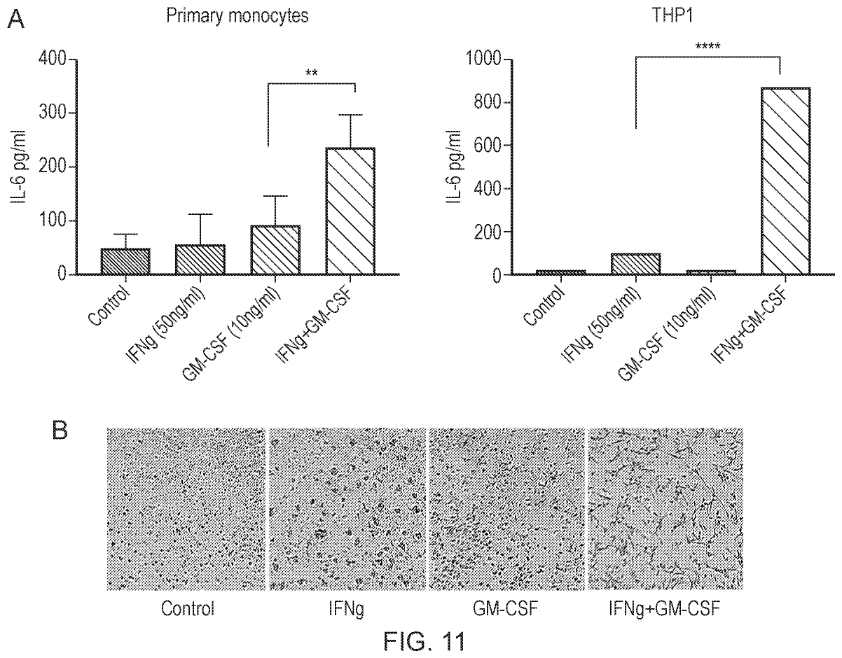

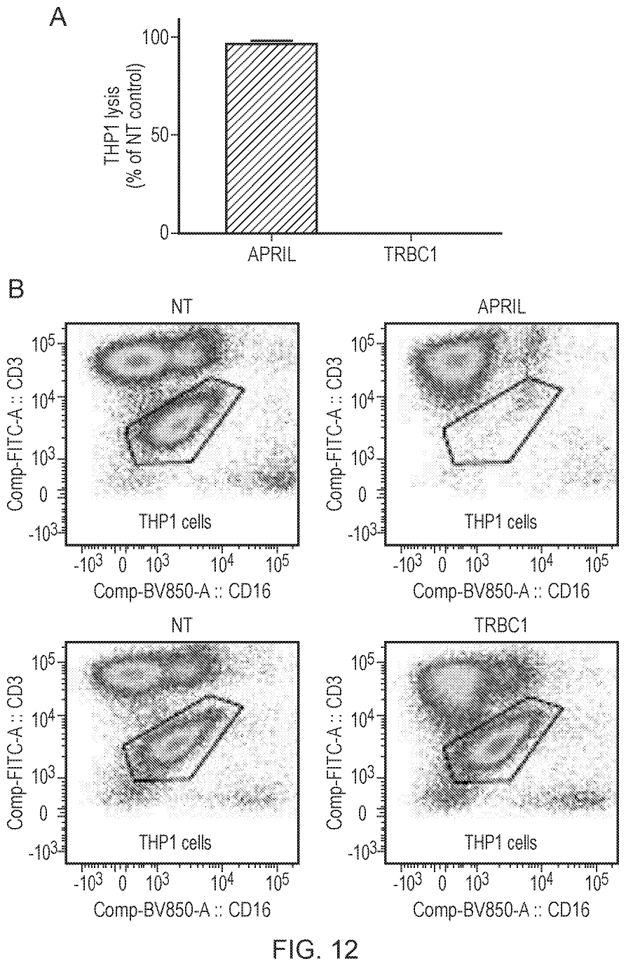

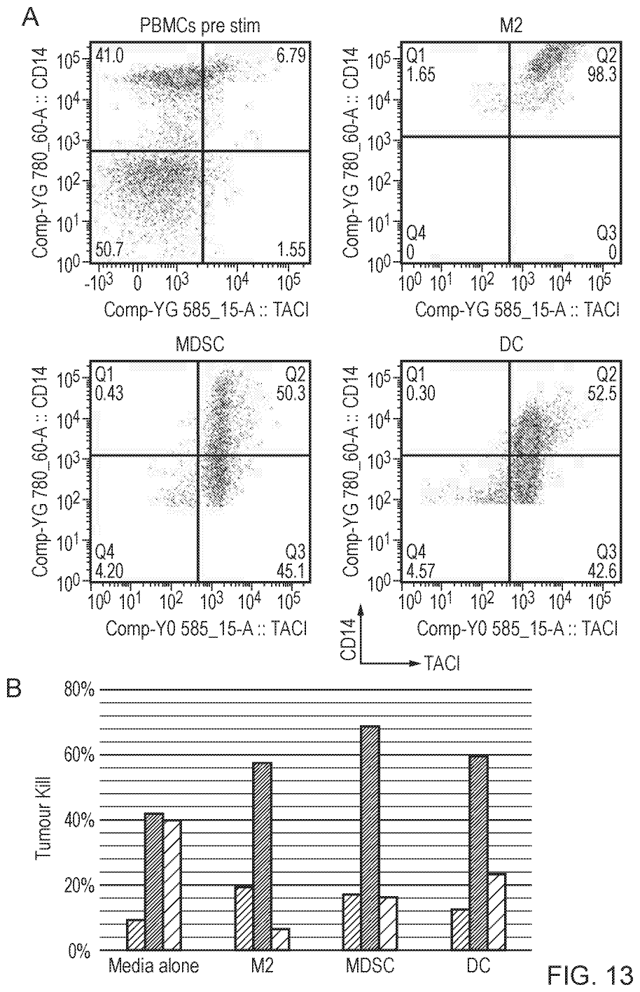

The TACI-specific antigen binding domain in the CAR or TanCAR of the present invention may lack all or part of a proliferation inducing ligand (APRIL). APRIL is the natural ligand for BCMA and TACI (see FIG. 4).

The BCMA-binding domain of the CAR of the invention may lack any part of a proliferation-inducing ligand (APRIL). APRIL is also known as TNFSF13.

The wild-type sequence of APRIL is available at UNIPROT/O75888 and is show below (SEQ ID No. 1). A truncated version of APRIL which retains BCMA and TACI binding but loses proteoglycan binding is shown as SEQ ID No. 2. SEQ ID No. 2 lacks the amino terminal 116 amino acids of the wild-type APRIL molecule shown as SEQ ID No. 1.

```
SEQ ID No. 1
                10 20 30 40
MPASSPFLLA PKGPPGNMGG PVREPALSVA LWLSWGAALG
                50 60 70 80
AVACAMALLT QQTELQSLRR EVSRLQGTGG PSQNGEGYPW
                90 100 110 120
QSLPEQSSDA LEAWENGERS RKRRAVLTQK QKKQHSVLHL
              130 140 150 160
VPINATSKDD SDVTEVMWQP ALRRGRGLQA QGYGVRIQDA
              170 180 190 200
GVYLLYSQVL FQDVTFTMGQ VVSREGQGRQ ETLFRCIRSM
              210 220 230 240
PSHPDRAYNS CYSAGVFHLH QGDILSVIIP RARAKLNLSP
              250
HGTFLGFVKL
SEQ ID No. 2
VLHLVPINATSKDDSDVTEVMWQPALRRGRGLQAQGYGVRIQDAGVYLLY
SQVLFQDVTFTMGQVVSREGQGRQETLFRCIRSMPSHPDRAYNSCYSAGV
FHLHQGDILSVIIPRARAKLNLSPHGTFLGFVKL
```

The TACI-specific antigen binding domain in the CAR or TanCAR of the present invention may lack any part of a proliferation inducing ligand (APRIL). For example it may lack any 10, 20, 30 or 40 amino acid-long stretch of consecutive amino acids from SEQ ID No. 1 or SEQ ID No. 2. It may lack the complete sequence shown as SEQ ID No. 2.

The TACI-specific antigen binding domain in the CAR or TanCAR may lack the BCMA binding domain of APRIL.

BAFF-R

The primary role of BAFF-R (UniProtKB: Q96RJ3), also known as BF3, is to mediate the survival and maturation of peripheral B cells. The protein encoded is a receptor for BAFF and is a type III transmembrane protein containing a single extracellular phenylalanine-rich domain. It is thought that this receptor is the principal receptor required for BAFF-mediated mature B-cell survival.

Studies conducted in BAFF-R deficient mice point to BAFF-R as the principle receptor transducing BAFF-mediated B lymphocyte survival signal.

The antigen binding domain of the first and/or second CAR, or the TanCAR which binds to BAFF-R may comprise a sequence derived from one of the commercially available anti-BAFF-R antibodies listed in the following table:

| Anti-BAFF-R antibody | Company |
|---|---|
| 11C1 | BioLegend |
| ab16232 | Abcam |
| ab5965 | Abcam |
| 9B9 | Adipogen |
| PA1391 | Boster Biological Technology |
| Anti-hBAFF-R | Axxora LLC |

BAFF

B cell activation factor of the TNF family (BAFF), also called BLyS, THANK, TALI-1 or zTNF4) is a cytokine expressed by myeloid cells that is considered to perform a proinflammatory function in B cell responses. Walters et al (2009, Journal of Immunology 182: 793-801) show that under specific conditions, BAFF also plays an anti-inflammatory role in T cell responses. Regulation of these roles is by modulation of cytokine responses. BAFF is mainly produced by myeloid cells, such as monocytes.

BAFF has been shown to be crucial in maintaining B lymphocyte homeostasis by facilitating the transition of B lymphocytes from T1 to T2 stage during maturation process. The pivotal role of BAFF in B lymphocyte survival and maturation has been demonstrated in vivo using a soluble BCMA molecule and in genetically engineered mice that do not express BAFF, which have decreased levels of mature B cells and severely impaired antibody response against T-dependent and T-independent antigens. In contrast, transgenic mice expressing high levels of BAFF were shown to have increased numbers of circulating mature B cells.

BAFF shows structural similarity and overlapping yet distinct receptor binding specificity with APRIL. The coordinate binding of BAFF to BCMA and/or TACI activates transcription factor NF-κB and increases the expression of pro-survival Bcl-2 family members (e.g. Bcl-2, Bcl-xL, Bcl-w, Mcl-1, A1) and the downregulation of pro-apoptotic factors (e.g. Bid, Bad, Bik, Bim, etc.), thus inhibiting apoptosis and promoting survival. This combined action promotes B cell differentiation, proliferation, survival and antibody production (as reviewed in Rickert R C et al., Immunol Rev (2011) 244 (1): 115-133).

The TACI-specific CAR or tanCAR of the invention may comprise an antigen-binding domain which comprises all or part of BAFF. The antigen-binding domain may, for example, comprise the TACI-binding domain of BAFF.

The 3D crystal structures of BAFF and TACI have been obtained, showing that the key domains in BAFF identified by TACI were the domains from 11e233 from Glu238 and from Asp 205 to Leu 211 (Wang et al., J Genet Syndr Gene Ther 2012, 3:3).

The amino acid sequence of BAFF is shown below

```
SEQ ID No. 3
                10 20 30 40
MDDSTEREQS RLTSCLKKRE EMKLKECVSI LPRKESPSVR
                50 60 70 80
SSKDGKLLAA TLLLALLSCC LTVVSFYQVA ALQGDLASLR
                90 100 110 120
AELQGHHAEK LPAGAGAPKA GLEEAPAVTA GLKIFEPPAP
              130 140 150 160
GEGNSSQNSR NKRAVQGPEE TVTQDCLQLI ADSETPTIQK
              170 180 190 200
GSYTEVPWLL SFKRGSALEE KENKILVKET GYFFIYGQVL
              210 220 230 240
YTDKTYAMGH LIQRKKVHVF GDELSLVTLF RCIQNMPETL
              250 260 270 280
PNNSCYSAGI AKLEEGDELQ LAIPRENAQI SLDGDVTFFG ALKIL
```

An antigen-binding domain of a CAR or tanCAR which binds BAFF-R, BCMA and/or TACI may comprise all or part of SEQ ID No. 3. For example, it may comprise a truncated version of SEQ ID No. 3 which retains BAFF-R, BCMA and/or TACI binding. It may comprise the BAFF-R, BCMA and/or TACI-binding site of BAFF. A TACI-binding antigen-binding domain may comprise residues 205-211 and 233-238 of SEQ ID No. 3. A TACI-binding antigen-binding domain may comprise residues 205-238 of SEQ ID No. 3.

BCMA

The B cell maturation target, also known as BCMA; TR17_HUMAN, TNFRSF17 (UniProt Q02223) is a transmembrane protein that is expressed in mature lymphocytes, e.g., memory B cells, plasmablasts and bone marrow plasma cells. BCMA is also expressed on myeloma cells. BCMA is a non-glycosylated type III transmembrane protein, which is involved in B cell maturation, growth and survival.

An antigen binding domain of a CAR or TanCAR which binds to BCMA may be any domain which is capable of binding BCMA. For example, the antigen binding domain may a sequence derived from one of the commercially available anti-BAFF-R antibodies listed in the following table:

| Anti-BCMA antibody | Company |
|---|---|
| ab5972 | Abcam |
| ab54834 | Abcam |
| SG1 | Seattle Genetic Inc |
| LS-B2728 | LS Biosciences |
| LS-C18716 | LS Biosciences |
| LS-C357630 | LS Biosciences |
| LS-C53526 | LS Biosciences |
| LS-C196740 | LS Biosciences |

The VH and VL sequences for three anti-BCMA antibodies are given below with CDR sequences underlined.

SEQ ID No. 4: antiBCMA Ab 1 VL
DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIKG
SEQ ID No. 5: antiBCMA Ab 1 VH
QIQLVQSGPELKKPGETVKISCKASGYTFTDYSINWVKRAPGKGLKWMGW
INTETREPAYAYDFRGRFAFSLETSASTAYLQINNLKYEDTATYFCALDY
SYAMDYWGQGTSVTVSS
SEQ ID No. 6: antiBCMA Ab 2 VL
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS
SEQ ID No. 7: antiBCMA Ab 2 VH
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR
SEQ ID No. 8: antiBCMA Ab 3 VL
EVQLVESGGGLVKPGRSLRLSCTASGFTFGDYALSWFRQAPGKGLEWVGV
SRSKAYGGTTDYAASVKGRFTISRDDSKSFAYLQMNSLKTEDTAVYYCCS
SGYSSGWTPFDYWGQGTLVTVSS
SEQ ID No. 9: antiBCMA Ab 3 VH
QSVLTQPPSASGTPGQRVTISCSGSSSNIGSNTVNWYQQLPGTAPKLLIF
NYHQRPSGVPDRFSGSKSGSSASLAISGLQSEDEADYYCAAWDDSLNGWV
FGGGTELTVLS

The antigen binding domain of a CAR or TanCAR which binds to BCMA may comprise the CDRs from antiBCMA Ab 1, 2 or 3, described above.

The antigen binding domain of a CAR or TanCAR which binds to BCMA may comprise the VH and/or VL sequence from antiBCMA Ab 1, 2 or 3, as described above, or a variant thereof which has at least 70, 80, 90 or 90% sequence identity, which variant retains the capacity to bind BCMA.

Transmembrane Domain

The CAR and/or the Tan CAR of the invention comprise a transmembrane domain which spans the membrane. It may comprise a hydrophobic alpha helix. The transmembrane domain may be derived from CD28, which gives good receptor stability.

The transmembrane domain may comprise the sequence shown as SEQ ID No. 10

SEQ ID No. 10
FWVLVVVGGVLACYSLLVTVAFIIFWV

Intracellular T Cell Signaling Domain (Endodomain)

The CAR of the invention may comprise or associate with an activating endodomain, the signal-transmission portion of the CAR. After antigen recognition, receptors cluster and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The endodomain of the CAR or TanCAR of the present invention may comprise the CD28 endodomain and OX40 and CD3-Zeta endodomain.

The endodomain may comprise:
(i) an ITAM-containing endodomain, such as the endodomain from CD3 zeta; and/or
(ii) a co-stimulatory domain, such as the endodomain from CD28; and/or
(iii) a domain which transmits a survival signal, for example a TNF receptor family endodomain such as OX-40 or 4-1BB.

An endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxLJI (SeQ ID NO. 51). Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

The sequence of some endodomains and co-stimulatory domains are given below.

(CD28 co-stimulatory endodomain)
SEQ ID No. 11
SKRSRLLHSDYMNMTPRRPGPTRKHYQPYAPPRDFAAYRS
(OX40 endodomain)
SEQ ID No. 12
RDQRLPPDAHKPPGGGSFRTPIQEEQADAHSTLAKI
(4-1BB endodomain)
SEQ ID No. 13
KRGRKKLLYIFKQPFMRPVQTTQEEDGCSCRFPEEEEGGCEL
(CD3zeta endodomain)
SEQ ID No. 14
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

The CAR may comprise a variant of any of the sequences shown as SEQ ID No. 11-14 having at least 80, 85, 90, 95, 98 or 99% sequence identity, provided that the variant sequence retains the capacity to induce T-cell signalling upon antigen recognition, i.e. provide the relevant activation/proliferation or survival signal to T cells.

A number of systems have been described in which the antigen recognition portion is on a separate molecule from the signal transmission portion, such as those described in WO015/150771; WO2016/124930 and WO2016/030691. The CAR or TanCAR of the present invention may lack a signalling endodomain.

The present invention also provides an antigen-binding component comprising a TACI-binding antigen-binding domain, and a transmembrane domain; capable of interacting with an intracellular signalling component comprising a signalling domain. The invention also provides a CAR signalling system comprising such an antigen-binding component and intracellular signalling component.

Signal Peptide

The cell of the present invention may comprise a signal peptide so that when the CAR or TanCAR is expressed inside a cell, the nascent protein is directed to the endoplasmic reticulum and subsequently to the cell surface, where it is expressed.

The core of the signal peptide may contain a long stretch of hydrophobic amino acids that tends to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The CAR of the invention may have the general formula:

Signal peptide—antigen binding domain—spacer domain—transmembrane domain—intracellular T cell signaling domain (endodomain).

The signal peptide may comprise the SEQ ID No. 15 or a variant thereof having 5, 4, 3, 2 or 1 amino acid mutations (insertions, substitutions or additions) provided that the signal peptide still functions to cause cell surface expression of the CAR.

SEQ ID No. 15:
METDTLLLWVLLLWVPGSTG

The signal peptide of SEQ ID No. 15 is compact and highly efficient. It is predicted to give about 95% cleavage after the terminal glycine, giving efficient removal by signal peptidase.

Spacer

The CAR and/or TanCAR of the present invention may comprise a spacer sequence to connect the antigen binding domain with the transmembrane domain and spatially separate the antigen binding domain from the endodomain. A flexible spacer allows to the antigen binding domain to orient in different directions to enable antigen binding. By way of example, the spacer sequence may connect to a TACI binding domain with the transmembrane domain and spatially separate the TACI binding domain from the endodomain. Alternatively, the spacer sequence may connect the BCMA binding domain with the transmembrane domain, and spatially separate the BCMA binding domain from the endodomain. Alternatively, the spacer sequence of the present invention may connect BAFF-R binding domain with the transmembrane domain, and spatially separate the BAFF-R binding domain from the endodomain.

The spacer sequence may, for example, comprise an IgG1 Fc region, an IgG1 hinge or a CD8 stalk, or a combination thereof. The spacer may alternatively comprise an alternative sequence which has similar length and/or domain spacing properties as an IgG1 Fc region, an IgG1 hinge or a CD8 stalk.

A human IgG1 spacer may be altered to remove Fc binding motifs. Examples of amino acid sequences for these spacers are given below:

(hinge-CH2CH3 of human IgG1)
SEQ ID No. 16
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):
SEQ ID No. 17
TTTPAPRPPTPAPTIASQPLSLRPEACRPAAGGAVHTRGLDFACDI
(human IgG1 hinge):
SEQ ID No. 18
AEPKSPDKTHTCPPCPKDPK
(IgG1 Hinge-Fc)
SEQ ID No. 19
AEPKSPDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK
(IgG1 Hinge - Fc modified to remove Fc receptor
recognition motifs)
SEQ ID No. 20
AEPKSPDKTHTCPPCPAPPVA*GPSVFLFPPKPKDTLMIARTPEVTCVVV
DVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDK
SRWQQGNVFSCSVMHEALHNHYTQKSLSLSPGKKDPK

Modified residues are underlined; * denotes a deletion.

Linker

The TanCAR of the second embodiment of the first aspect of the present invention comprises a linker sequence to link the two antigen binding domains in tandem. By way of example, the two antigen binding domains linked together by the linker may bind the antigen TACI and the antigen BCMA. Alternatively, the two antigen binding domains linked together by the linker may bind the antigen TACI and the antigen BAFF-R. An example of a highly flexible linker is given below:

SEQ ID No. 21
(Gly-Ser Linker)
SGGGS

Nucleic Acid Sequence

The second aspect of the invention relates to a nucleic acid sequence which codes for a TanCAR.

The nucleic acid, when expressed by a target cell, causes the encoded TanCAR to be expressed at the cell-surface of the target cell.

The nucleic acid sequence may be RNA or DNA, such as cDNA.

Nucleic Acid Construct

The present invention provides a nucleic acid construct which comprises two or more nucleic acid sequences. For example, the nucleic acid construct may encode:

a first CAR which binds TACI and a second CAR which binds an antigen other than TACI;

a first CAR which binds TACI, a second CAR which binds an antigen other than TACI, and a suicide gene; or

a TanCAR and a suicide gene

The nucleic acid, when expressed by a target cell, causes the encoded polypeptides to be expressed independently within or at the surface of the target cell.

The nucleic acid sequences in the nucleic acid construct may be separated by a “coexpression” sequence which enables the two polypeptides, once translated, to be expressed separately in or on the cell.

In the structure above, “coexpr” is a nucleic acid sequence enabling co-expression of the two polypeptides. It may be a sequence encoding a cleavage site, such that the nucleic acid construct produces comprises two or more polypeptides joined by a cleavage site(s). The cleavage site may be self-cleaving, such that when the polypeptide is produced, it is immediately cleaved into individual polypeptides without the need for any external cleavage activity.

The cleavage site may be any sequence which enables the two or more polypeptides to become separated.

The term “cleavage” is used herein for convenience, but the cleavage site may cause the polypeptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′ (SEQ ID No. 52)) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond) (SEQ ID No. 53). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

| | |
|---|---|
| YHADYYKQRLIHDVEMNPGP | (SEQ ID No. 22) |
| HYAGYFADLLIHDIETNPGP | (SEQ ID No. 23) |
| QCTNYALLKLAGDVESNPGP | (SEQ ID No. 24) |
| ATNFSLLKQAGDVEENPGP | (SEQ ID No. 25) |
| AARQMLLLLSGDVETNPGP | (SEQ ID No. 26) |
| RAEGRGSLLTCGDVEENPGP | (SEQ ID No. 27) |
| TRAEIEDELIRAGIESNPGP | (SEQ ID No. 28) |
| TRAEIEDELIRADIESNPGP | (SEQ ID No. 29) |
| AKFQIDKILISGDVELNPGP | (SEQ ID No. 30) |
| SSIIRTKMLVSGDVEENPGP | (SEQ ID No. 31) |
| CDAQRQKLLLSGDIEQNPGP | (SEQ ID No. 32) |
| YPIDFGGFLVKADSEFNPGP | (SEQ ID No. 33) |

The cleavage site may comprise the 2A-like sequence shown as SEQ ID No. 27 (RAEGRGSLLTCGDVEENPGP).

Suicide Genes

Since T-cells engraft and are autonomous, a means of selectively deleting CAR T-cells in patients is desirable. Suicide genes are genetically encodable mechanisms which result in selective destruction of infused T-cells in the face of unacceptable toxicity. The earliest clinical experience with suicide genes is with the Herpes Virus Thymidine Kinase (HSV-TK) which renders T-cells susceptible to Ganciclovir. HSV-TK is a highly effective suicide gene. However, pre-formed immune responses may restrict its use to clinical settings of considerable immunosuppression such as haploidentical stem cell transplantation. Inducible Caspase 9 (iCasp9) is a suicide gene constructed by replacing the activating domain of Caspase 9 with a modified FKBP12. iCasp9 is activated by an otherwise inert small molecular chemical inducer of dimerization (CID). iCasp9 has been recently tested in the setting of haploidentical HSCT and can abort GvHD. The biggest limitation of iCasp9 is dependence on availability of clinical grade proprietary CID. Both iCasp9 and HSV-TK are intracellular proteins, so when used as the sole transgene, they have been co-expressed with a marker gene to allow selection of transduced cells.

An iCasp9 suicide gene may comprise the sequence shown as SEQ ID No. 34 or a variant thereof having at least 80, 90, 95 or 98% sequence identity.

SEQ ID No. 34
MLEGVQVETISPGDGRTFPKRGQTCVVHYTGMLEDGKKVDSSRDRNKPFK
FMLGKQEVIRGWEEGVAQMSVGQRAKLTISPDYAYGATGHPGIIPPHATL
VFDVELLKLESGGGSGVDGFGDVGALESLRGNADLAYILSMEPCGHCLII
NNVNFCRESGLRTRTGSNIDCEKLRRRFSSLHFMVEVKGDLTAKKMVLAL
LELAQQDHGALDCCVVVILSHGCQASHLQFPGAVYGTDGCPVSVEKIVNI
FNGTSCPSLGGKPKLFFIQACGGEQKDHGFEVASTSPEDESPGSNPEPDA
TPFQEGLRTFDQLDAISSLPTPSDIFVSYSTFPGFVSWRDPKSGSWYVET
LDDIFEQWAHSEDLQSLLLRVANAVSVKGIYKQMPGCFNFLRKKLFFKTS
AS

WO2013/153391 describes a marker/suicide gene known as RQR8 which can be detected with the antibody QBEnd10 and expressing cells lysed with the therapeutic antibody Rituximab.

The suicide gene may comprise the sequence shown as SEQ ID No. 35 or a variant thereof having at least 80, 90, 95 or 98% sequence identity.

SEQ ID No. 35
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV

The suicide gene may be expressed as a single polypeptide with the CAR or TanCAR, for example by using a self-cleaving peptide between the two sequences.

Vector

The present invention also provides a vector which comprises a nucleic acid sequence or a nucleic acid construct according to the present invention. Such a vector may be used to introduce the nucleic acid sequence or construct into a host cell so that it expresses and produces one or more CAR(s), a tanCAR and/or a suicide gene.

The vector may, for example, be a plasmid or synthetic mRNA or a viral vector, such as a retroviral vector or a lentiviral vector.

The vector may be capable of transfecting or transducing an effector cell.

Cell

The present invention provides a cell which expresses one or more CARs/TanCARs optionally together with a suicide gene

The cell may comprise a nucleic acid sequence or construct according to the invention.

The host cell may be an immune cell such as a T cell or an Natural Killer (NK) cell. The host cell may be a cytolytic immune cell.

For cells other than T cells, such as NKT cells, the signalling domain(s) of the CAR may be different from a T cell signalling domain and may be tailored for that particular cell type.

The cell may made by transducing or transfecting a cell with CAR-encoding or TanCAR-encoding nucleic acid.

The cell may be an ex vivo T cell. The T cell may be from a peripheral blood mononuclear cell (PBMC) sample. T cells may be activated and/or expanded prior to being transduced with a CAR- or TanCAR-encoding nucleic acid, for example by treatment with an anti-CD3 monoclonal antibody.

Pharmaceutical Composition

The present invention also relates to a pharmaceutical composition which comprises a plurality of cells according to the first aspect of the invention.

The present invention also provides a pharmaceutical composition comprising a mixture of the following:

(i) a first cell population expressing a first CAR; and

(ii) a second cell population expressing a second CAR

in which the first and second CARs bind different antigens and the first CAR binds to Transmembrane activator and calcium modulator and cyclophilin ligand interactor (TACI).

The second CAR may bind to BCMA.

The pharmaceutical compositions of the invention may also comprise a pharmaceutically acceptable carrier, diluent or excipient, and optionally one or more further pharmaceutically active polypeptides and/or compounds. Such a formulation may, for example, be in a form suitable for intravenous infusion.

Method of Treatment

The cells of the present invention may capable of killing cancer cells, such as late stage B-cell malignancies.

The cells may either be created ex vivo either from a patient's own peripheral blood (1st party), or in the setting of a haematopoietic stem cell transplant from donor peripheral blood (2nd party), or peripheral blood from an unconnected donor (3rd party). Alternatively, the cells may be derived from ex vivo differentiation of inducible progenitor cells or embryonic progenitor cells. In these instances, the cells are generated by introducing DNA or RNA coding for the CAR or TanCAR by one of many means including transduction with a viral vector, transfection with DNA or RNA.

The present invention also provides a method for treating and/or preventing a disease, which comprises the following steps:

(i) administration of a first cell population which expresses a first CAR

(ii) administration of a second cell population which expresses a second CAR

wherein the first and second CARs bind different antigens and wherein the first or second CAR binds to Transmembrane activator and calcium modulator and cyclophilin ligand interactor (TALI).

The TACT-CAR cell population may be administered to the patient before, after or simultaneously with administration of the cell population with binding specificity for an antigen other than TACT.

There is also provided a kit for use in such a method, which comprises:

(i) a first cell population which expresses a first CAR

(ii) a second cell population which expresses a second CAR

wherein the first and second CARs bind different antigens and wherein the first CAR binds to Transmembrane activator and calcium modulator and cyclophilin ligand interactor (TACT).

The first and second cell populations may be provided separately in the kit (e.g in separate containers) for subsequent, separate or simultaneous administration. The first cell population may be administered to the patient before, after or at the same tme as the second cell population.

The method of the present invention may be for treating a cancerous disease, in particular a plasma cell disorder or a B cell disorder which correlates with enhanced TACT/BCMA expression.

Plasma cell disorders include plasmacytoma, plasma cell leukemia, multiple myeloma, macroglobulinemia, amyloidosis, Waldenstrom's macroglobulinemia, solitary bone plasmacytoma, extramedullar plasmacytoma, osteosclerotic myeloma (POEMS Syndrome) and heavy chain diseases as well as the clinically unclear monoclonal gammopathy of undetermined significance/smoldering multiple myeloma.

The disease may be multiple myeloma.

Examples for B cell disorders which correlate with elevated TACl/BCMA expression levels are CLL (chronic lymphocytic leukemia) and non-Hodgkins lymphoma (NHL).

A method for the treatment of disease relates to the therapeutic use of a pharmaceutical composition of the invention. In this respect, the pharmaceutical composition may be administered to a subject having an existing disease or condition in order to lessen, reduce or improve at least one symptom associated with the disease and/or to slow down, reduce or block the progression of the disease. The method of the invention may cause or promote cell mediated killing of TACI-expressing or TACl/BCMA-expressing cells, such as plasma cells.

Inhibition of Cytokine Release Syndrome

The thirteenth aspect of the invention relates to a method for avoiding and/or reducing cytokine release syndrome in a subject.

The present inventors have found that TACI is expressed on myeloid cells such as monocytes and macrophages which secrete pro-inflammatory cytokines such as IL-6. These cytokines are associated with cytokine release syndrome. It is therefore possible to prevent, reduce or treat CRS in a patient by targeting monocytes and macrophages via TACI.

The secretion of cytokines such as IL-6 is also thought to contribute to the hostile microenvironment associated with cancers, in particular solid-tumours. TACI may also be targeted to prevent or reduce cytokine secretion by myeloid cells in order to ameliorate the microenvironment and make it more conducive to an effective anti-cancer immunological response.

The anti-TACI agent may inhibit cytokine secretion by myeloid cells in the subject. The anti-TACI agent may kill myeloid cells in the subject, for example by apoptosis or other mechanisms of cell death. The anti-TACI agent may ameliorate the tumour microenvironment.

TACI may be targeted using an anti-TACI antibody. The agent may, for example be an anti-TACI antibody-drug conjugate (ADC, a bispecific antibody or a bispecific T-cell engager (BiTE).

Alternatively TACI may be targeted using an anti-TACI chimeric antigen receptor (CAR). The CAR may comprise a binding domain based on APRIL, the natural ligand for TACI, as described in WO2015/052538; or it may comprise and anti-TACI scFv.

The anti-TACI CAR comprise two separate components: an antigen-binding component, which comprises an antigen-binding domain and a transmembrane domain; and an intracellular signalling component which comprises an endodomain. The antigen binding component and intracellular signalling component associate at the cell surface to form a functional CAR complex.

The antigen binding component and intracellular signalling component may associate only in the presence or absence of certain molecule, as described in WO2015/150771 and WO2016/030691 respectively. The molecule, for example tetracycline, may cause dissociation of the antigen binding component and intracellular signalling component, reducing or stopping CAR function.

The anti-TACI agent may be or comprise a cell according to the first aspect of the invention.

Where the cell comprises an OR gate (the first embodiment of the first aspect of the invention) or a TanCAR (the second embodiment of the first aspect of the invention), the second antigen binding domain may bind any other antigen, for example any other tumour associated antigen.

The second antigen-binding domain may bind one of the following tumour associated antigens:

| Cancer type | TAA |
|---|---|
| Diffuse Large B-cell Lymphoma | CD19, CD22, CD20 |
| Acute lymphoblastic Leukemia | |
| T-cell malignancies | TRBC1, TRBC2 |
| Breast cancer | ErbB2, MUC1 |
| AML | CD13, CD33 |
| Neuroblastoma | GD2, NCAM |
| B-CLL | CD19, CD52 |
| Colorectal cancer | Folate binding protein, CA-125 |

CRS may be reduced or avoided during the treatment of a cancer. The second antigen binding domain may target an antigen associated with that cancer. The cancer may be multiple myeloma, a haematological malignancy or a solid tumour. The cancer may be one of the ones listed in the table above.

The anti-TACI agent may be used for treating CRS. A method for treating CRS relates to the therapeutic use of the agent (e.g. cells) of the present invention. Herein the agent may be administered to a subject having existing CRS or CRS-associated condition in order to lessen, reduce or improve at least one symptom associated with CRS and/or to slow down, reduce or block the progression of CRS.

The subject may have previously been treated with CAR-expressing cells.

The method for preventing CRS relates to the prophylactic use of the agent (e.g. cells) of the present invention. For this use, the agent may be administered to a subject who is not showing any symptoms of CRS to prevent or impair the cause of CRS or to reduce or prevent development of at least one symptom associated with CRS. The subject may be about to receive an immunotherapy, such as a CAR-based treatment.

For prophylactic use, the anti-TACI agent may be administered before the CAR-based therapy, for example in a pre-conditioning regimen. Alternatively, the anti-TACI agent may co-administered with the CAR-based therapy.

The invention will now be further described by way of Examples, which are meant to serve to assist one of ordinary skill in the art in carrying out the invention and are not intended in any way to limit the scope of the invention.

### EXAMPLES

### Example 1

### Expression of BCMA on Surface of Myeloma Cells

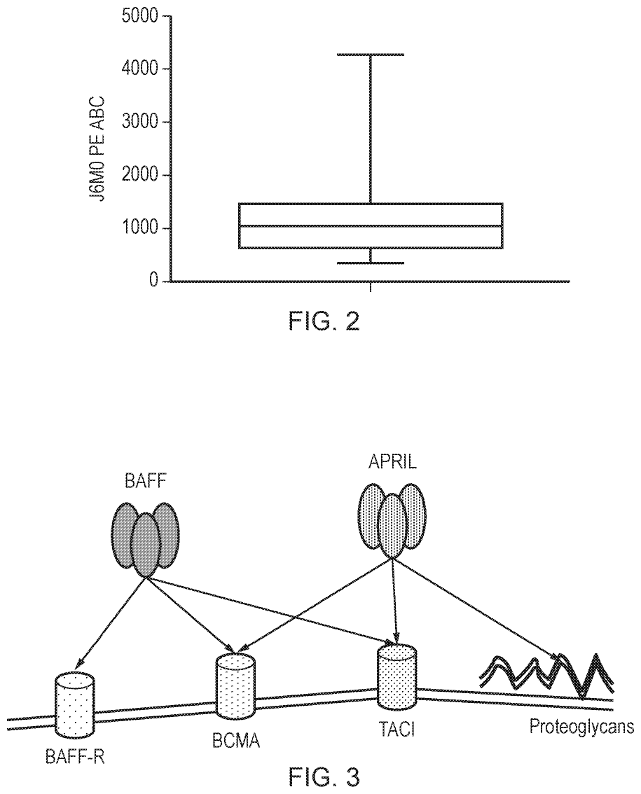

Primary myeloma cells were isolated by performing a CD138 immunomagnetic selection on fresh bone marrow samples from Multiple myeloma patients that were known to have frank disease. These cells were stained with the BCMA specific J6MO mAb (GSK) which was conjugated to PE. At the same time, a standard of beads with known numbers of binding sites was generated using the PE Quantibrite bead kit (Becton Dickenson) as per the manufacturer's instructions. The BCMA copy number on myeloma cells was derived by correlating the mean-fluorescent intensity from the myeloma cells with the standard curve derived from the beads. It was found that the range of BCMA copy number on a myeloma cell surface is low: at 348.7-4268.4 BCMA copies per cell with a mean of 1181 and a median of 1084.9 (FIG. 2). This is considerably lower than e.g. CD19 and GD2, classic targets for CARs.

### Example 2

### Design and Generation of TACI CARs

Immunization and Hybridoma Generation

The gene coding for human TACI was cloned in the vector pVAC2. 5 BalBC mice were immunized with plasmid DNA encoding TACI adsorbed to gold nanoparticles. A GeneGun™ (Biorad) system was used to deliver the coated gold nanoparticles intramuscularly. The mice were boosted 3 times over the course of 21 days. Test bleeds from the mice were screened for titres of anti-TACI antibodies by ELISA and flow cytometry.

Five mice with TACI positive sera were selected for a final immunization boost before the spleens were harvested for B cell isolation and hybridoma production. Hybridoma fusions of 10×96-well plates with lymphocytes from the selected mice were performed. Hybridoma supernatants were screened for reactive anti-TACI antibodies by ELISA screening against recombinant human TACI purified protein (Peprotech, 310-17), representative data is shown in FIG. 6a. ELISA positive hybridoma supernatants were tested by flow cytometry, on SupT1 cells engineered to overexpress TACI. As a control BCMA expressing SupT1 cells were used, FIG. 6b. Candidate hybridomas were expanded.

Hybridoma Expansion and Cloning

Four parental clone hybridomas expressing the strongest and most specific anti-TACI response by flow cytometry (2H6, 2G2 1G6, 4B11) were identified, expanded, and stocks cloned to generate monoclonal antibody secreting hybridomas. Hybridoma clones were obtained by limiting dilution.

Antibody Sequencing from Monoclonal Hybridoma Cell Lines

Total RNA was isolated from monoclonal hybridoma cells using TRIzol® Reagent according to the manufacturer's instructions. The total RNA was analyzed by agarose gel electrophoresis and the concentration assessed using a NanoDrop2000C. Total RNA was reverse-transcribed into cDNA using either isotype-specific anti-sense primers or universal primers using PrimeScript™ 1st Strand cDNA Synthesis Kit according to manufacturer's instructions. The antibody fragments of VH and VL were amplified using the 5′RACE PCR method. DNA fragments were cloned blunt ended into vectors containing T overhangs (TOPO—Thermofisher). 5 colonies for each of the heavy and light chains were sequenced and a consensus sequence was obtained. The sequences for the clones, together with the CDR sequences for the VH and VL regions, are shown above on pages 17-19.

Cloning of TACI Binding Antibodies in Soluble Fc Tagged and CAR Formats.

Variable heavy and light chains were cloned into scFv format using standard molecular biology techniques. aTACI scFvs were tested for their ability to bind to cells by flow cytometry and for their binding kinetics by BIAcore, FIG. 8.

ScFv antibodies were engineered as in chimeric antigen receptor format. To demonstrate expression in CAR format, the scFv of each CAR was cloned in a single open-reading frame with human Hinge, TYRP transmembrane domain and CD28 OX40 and CD3 zeta endo-domains in the SFG retroviral vector, FIG. 7a. T-cells were transduced with this vector and CARs were detected on the T-cells surface expressing the cassette by staining with recombinant TACI fused to the Fc domain of a rabbit IgG1.

### Example 3

### Killing of Target Cells by TACI-CAR T Cells

Retroviruses were produced by transient transfection of 293T cells with plasmids encoding the CARs, gag/pol and the envelope protein RD114. After 3 days the supernatants were harvested and used to transduce PHA/IL2-activated PBMCs with equal titres of retrovirus on retronectin-coated plates. Six days post-transduction CAR-expression was confirmed by flow cytometry and PBMCs were co-cultured in a 1: 1 ratio with either TACI+BFP SupT1 cells or BCMA+BFP SupT1 cells. Target cell killing was assayed after one and three days. Also after one and three days, supernatants were removed and interferon-γ levels were assayed by ELISA. The results are shown in FIGS. 7b and c.

### Example 4

### The Effect of TACI-CAR on IL-6 Production by Myeloid Cells

Myeloid cells, both cell lines and primary cells isolated from peripheral blood, are treated with conditioned media from CAR-T cells exposed to target cells in vitro, to investigate the secretion of IL-6 by myeloid cells in response to cytokines produced by activated CAR-T cells.

TACI expression by myeloid cells is also investigated before and after treatment with conditioned media from CAR-T cells exposed to target cells.

A co-culture is established with CAR-T cells, target cells and myeloid cells, to investigate the effect of TACI-mediated CAR-T cell killing of myeloid cells on IL-6 production. Two different types of CAR-T cells are tested: APRIL CAR-expressing T cells (see FIG. 9A) and CD19/22 CAR-expressing T cells (see FIG. 9B). APRIL CAR comprises a truncated version of APRIL as antigen binding domain. The sequence for this is given above as SEQ ID No. 2. Truncated APRIL retains BCMA and TACI binding, but no longer has the capacity for proteoglycan binding. CD19/CD22 CAR T cells express two CARS, one against CD19 and one against CD22. CD19/22 CAR-expressing T cells do not kill myeloid cells and act as a negative control.

The potential for Trametinib, an MEK inhibitor, to inhibit IL-6 by myeloid cells in the presence of cytokines secreted by CAR-T cells upon activation with target cells is also investigated.

### Example 5

### APRIL Based CARs are able to Kill IL-6-Producing Monocytes

CRS is one of the main adverse effects of CART therapy caused by high levels of cytokines such as IL-6. To investigate a potential mechanism of CRS triggering in patients, we analysed the concentration of pro-inflammatory cytokines in the supernatants from a 48 h co-culture of APRIL CART cells with MM1s target cells seeded at 1:1 ratio. This analysis performed by Luminex Multiplex assay revealed GM-CSF and IFNg to be the two most abundant cytokines among the tested cytokine panel (FIG. 10A). These cytokines were not present in the supernatants from co-culture of non-transduced T cells and MM1s cells (FIG. 10B). To investigate the effect of GM-CSF and IFNg on IL-6 production in monocytes, human primary monocytes isolated from 3 healthy donors or THP1 monocyte-like cells were cultured for 48 h either alone or in the presence of IFNg (50 ng/ml) or GM-CSF (10 ng/ml) or the combination of both. Following the incubation, IL-6 levels were measured in the culture media by ELISA. The results are shown in FIG. 11. Both primary monocytes and THP1 cells were found to have increased production of IL-6 in response to the combination of IFNg and GM-CSF when compared to the untreated control or the single cytokine treatment. Human primary monocytes were also found to show distinct morphological changes in response to combination of IFNg and GM-CSF following 48 h incubation (FIG. 11B). In order to investigate the cytotoxic activity of APRIL CAR-T cells against monocytes, APRIL CARTs were co-cultured with THP1 cells for 72 h. Control CAR-expressing T cells were also tested in which the CAR was specific for the T-cell antigen TRBC1. The APRIL CAR-T cells but not the non-transduced or TRBC1 CAR-T cells showed a highly cytotoxic effect against these monocyte cells as determined by FACS (FIG. 12).

### Example 6

### APRIL CAR Gives Superior Killing of Target Cells in the Presence of Monocyte Derive Suppressor Cells

Survival of malignant plasma cells are supported by interactions with the bone marrow microenvironment (Kawano et al (2015) Immunol. Rev. 263:160-172). These interactions include tumour associated macrophages, myeloid-derived suppressor cells (MDSCs) and dendritic cells (DCs). These cell types both support the myeloma cell population and suppress tumour-directed immune responses. Patients with high levels of bone marrow infiltration with bone marrow macrophages have poor prognosis (Suyani et al (2013) Ann Hematology 92:669-677) and the number of MDSCs is significantly increased in the peripheral blood and the bone marrow of multiple myeloma patients (Gorgun et al (2013) Blood 121:2975-2987).

In the same way the that immunosuppressive bone marrow microenvironment has been shown to reduce endogenous T cell activity, adoptively transferred CAR-T cells are affected by the immunosuppressive cells in the bone marrow microenvironment of myeloma patients. MDSCs, macrophages and DCs are derived from monocytes so we developed an in vitro system to assess CAR mediated tumour cytolysis in the presence of these monocyte derivatives cultured from autologous peripheral blood mononuclear cells (PBMCs).

Monocytes were isolated from PBMCs using pan monocyte isolation microbeads (Miltenyl) and cultured in cytokines for a week to derive M2 monocytes, MDSCs and DC. These cells have a suppressive phenotype akin to tumour infiltrating macrophages found in the bone marrow of myeloma patients (Gutiérrez-González et al (2016) Blood 128:2241-2252). CART cells derived from the same donor were then co-cultured with a human myeloma cell line (HMCL) MM.1s in the absence or presence of these suppressive cell types.

Two types of CAR were tested, APRIL CAR and an anti-BCMA CAR comprising an scFv-based antigen binding domain derived from an anti-BCMA monoclonal antibody (BCMAscFv CAR). In this system, significantly attenuated tumour kill was seen by the BCMAscFv CAR in the presence of M2 monocytes, MDSCs and DCs (FIG. 13). By contrast, APRIL CAR mediated killing was maintained in the presence of monocyte derived suppressor cells.

By FACS analysis, we established that M2 monocytes, MDSCs and DCs express TACI (FIG. 13a). The dual targeting capabilities of APRIL CAR T cells allows the direct targeting of TACI expressing immunosupressive cells. Thus while these monocyte derivatives suppress the tumour kill by CAR T cells targeting BCMA alone, APRIL CAR mediated killing is maintained.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell comprising first and second chimeric antigen receptors (CARs), which bind to different antigens, wherein the first CAR binds to Transmembrane activator and calcium modulator and cyclophilin ligand interactor (TACI).

2. A cell according to claim 1 where the second CAR binds BCMA.

3. A cell comprising a tanCAR comprising first and second antigen-binding domains which bind to different antigens, wherein the first antigen binding domain binds the antigen Transmembrane activator and calcium modulator and cyclophilin ligand interactor (TACI).

4. A cell according to claim 3 where the second antigen binding domain binds to BCMA.

5. A nucleic acid sequence encoding a tanCAR as defined in claim 3 or 4.

6. A nucleic acid sequence according to claim 5, which has the following structure:
AgB1-linker-AgB2-spacer-TM-endo
in which
AgB1 is a nucleic acid sequence encoding the first antigen-binding domain of the tanCAR;
linker is a nucleic acid sequence encoding a linker of the tanCAR;
AgB2 is a nucleic acid sequence encoding the second antigen binding domain of the tanCAR;
spacer is a nucleic acid sequence encoding a spacer of the tanCAR;
TM is a nucleic acid sequence encoding a transmembrane domain of the tanCAR;
endo is a nucleic acid sequence encoding an endodomain of the tanCAR;
which nucleic acid sequence, when expressed in a cell, encodes a polypeptide expressing the first and second antigen binding domains in tandem at the cell surface.

7. A nucleic acid sequence according to claim 6, wherein linker encodes a sequence comprising a Gly-Ser flexible linker.

8. A nucleic acid sequence according to any of claims 5 to 7 wherein alternative codons are used in regions of sequence encoding the same or similar amino acid sequences, in order to avoid homologous recombination.

9. A nucleic acid construct which encodes a first CAR as defined in claim 1 or 2; and a second CAR as defined in claim 1 or 2.

10. A nucleic acid construct according to claim 9, which has the following structure:
AgB1-spacer1-TM1-endo1-coexpr-AgB2-spacer2-TM2-endo2
in which
AgB1 is a nucleic acid sequence encoding the antigen-binding domain of the first CAR;
spacer1 is a nucleic acid sequence encoding a spacer of the first CAR;
TM1 is a nucleic acid sequence encoding a transmembrane domain of the first CAR;
endo1 is a nucleic acid sequence encoding an endodomain of the first CAR;
coexpr is a nucleic acid sequence enabling co-expression of the first and second CARs
AgB2 is a nucleic acid sequence encoding the antigen-binding domain of the second CAR;
spacer2 is a nucleic acid sequence encoding the spacer of the second CAR;
TM2 is a nucleic acid sequence encoding the transmembrane domain of the second CAR;
endo2 is a nucleic acid sequence encoding the endodomain of the second CAR;
which nucleic acid construct, when expressed in a cell, encodes a polypeptide which is cleaved at the cleavage site such that the first and second CARs are co-expressed at the cell surface.

11. A nucleic acid construct according to claim 10, wherein coexpr encodes a sequence comprising a self-cleaving peptide.

12. A nucleic acid construct according to any of claims 9 to 11, wherein alternative codons are used in regions of sequence encoding the same or similar amino acid sequences, in order to avoid homologous recombination.

13. A vector comprising a nucleic acid sequence according to any of claims 5 to 8, or a nucleic acid construct according to any of claims 9 to 12.

14. A retroviral vector or a lentiviral vector or a transposon according to claim 13.

15. A kit which comprises
(i) a first nucleic acid sequence encoding a first CAR as defined in claim 1 or 2, which nucleic acid sequence has the following structure:
AgB1-spacer1-TM1-endo1
in which
AgB1 is a nucleic acid sequence encoding the antigen-binding domain of the first CAR;
spacer 1 is a nucleic acid sequence encoding the spacer of the first CAR;
TM1 is a nucleic acid sequence encoding the transmembrane domain of the first CAR;
endo 1 is a nucleic acid sequence encoding the endodomain of the first CAR; and
(ii) a second nucleic acid sequence encoding a second CAR as defined in claim 1 or 2, which nucleic acid sequence has the following structure:
AgB2-spacer2-TM2-endo2
in which
AgB2 is a nucleic acid sequence encoding the antigen-binding domain of the second CAR;
spacer 2 is a nucleic acid sequence encoding the spacer of the second CAR;
TM2 is a nucleic acid sequence encoding the transmembrane domain of the second CAR;
endo 2 is a nucleic acid sequence encoding the endodomain of the second CAR.

16. A kit comprising: a first vector which comprises the first nucleic acid sequence as defined in claim 15; and a second vector which comprises the second nucleic acid sequence as defined in claim 15.

17. A kit according to claim 16, wherein the vectors are integrating viral vectors or transposons.

18. A method for making a cell according to any of claims 1 to 4, which comprises the step of introducing: a nucleic acid sequence according to any of claims 5 to 8; a nucleic acid construct according to any of claims 10 to 12; a vector according to claim 13 or 14 or a kit of sequences or vectors according to any of claims 15 to 17, into a cell.

19. A method according to claim 18, wherein the cell is from a sample isolated from a subject.

20. A pharmaceutical composition comprising a plurality of cells according to any of claims 1 to 4.

21. A pharmaceutical composition comprising:
(i) a first cell population expressing a first CAR; and
(ii) a second cell population expressing a second CAR
wherein the first and second CARs bind different antigens and the first CAR binds to Transmembrane activator and calcium modulator and cyclophilin ligand interactor (TACI).

22. A method for treating and/or preventing a disease, which comprises the step of administering a pharmaceutical composition according to claim 20 or 21 to a subject.

23. A method for treating and/or preventing a disease, which comprises the following steps:
(i) administration of a first cell population which expresses a first CAR
(ii) administration of a second cell population which expresses a second CAR
wherein the first and second CARs bind different antigens and wherein the first CAR binds to Transmembrane activator and calcium modulator and cyclophilin ligand interactor (TALI).

24. A method according to claim 22 or 23, which comprises the following steps:
(i) isolation of a cell-containing sample from a subject;
(ii) transduction or transfection of the cells with: a nucleic acid sequence according to any of claims 5 to 8; a nucleic acid construct according to any of claims 10 to 12; a vector according to claim 13 or 14 or a kit of sequences or vectors according to any of claims 15 to 17; and
(iii) administering the cells from (ii) to the subject.

25. A kit for use in a method according to claim 23, which comprises:
(i) a first cell population which expresses a first CAR
(ii) a second cell population which expresses a second CAR
wherein the first and second CARs bind different antigens and wherein the first CAR binds to Transmembrane activator and calcium modulator and cyclophilin ligand interactor (TALI).

26. A method according to any of claims 22 to 24, wherein the disease is a cancer.

27. A method according to claim 26 wherein disease is a mature B cell malignancy.

28. A method according to claim 26 or 27, wherein the disease is multiple myeloma.

29. A pharmaceutical composition according to claim 20 or 21 for use in treating and/or preventing a disease.

30. The use of a cell according to any of claims 1 to 4 in the manufacture of a medicament for treating and/or preventing a disease.

31. A tanCAR comprising a first and second antigen-binding domain which bind to different antigens, wherein the first antigen binding domain binds the antigen Transmembrane activator and calcium modulator and cyclophilin ligand (CAML) interactor (TACI).

32. A method for avoiding and/or reducing cytokine release syndrome in a subject which comprises the step of administering an anti-TACI agent to the subject.

33. A method according to claim 32, wherein the anti-TACI agent is an anti-TACI antibody.

34. A method according to claim 32, wherein the anti-TACI agent is a cell which expresses and anti-TACI chimeric antigen receptor (CAR).

35. A method according to claim 34, wherein the anti-TACI CAR comprises two separate components: an antigen-binding component, which comprises an antigen-binding domain and a transmembrane domain; and an intracellular signalling component which comprises an endodomain in which the antigen binding component and intracellular signalling component associate at the cell surface to form a functional CAR complex.

36. A method according to claim 34, wherein the anti-TACI CAR has an antigen binding domain which comprises the BCMA binding domain from APRIL.

37. A method according to claim 34, wherein the anti-TACI agent is a cell according to any of claims 1 to 4.

38. A method according to any of claims 32 to 37, wherein the subject is undergoing or about to receive CAR-based immunotherapy.

39. A method according to any of claims 32 to 38, wherein the anti-TACI agent inhibits cytokine secretion by myeloid cells in the subject.

40. A method according to any of claims 32 to 38, wherein the anti-TACI agent kills myeloid cells in the subject.

41. An anti-TACI agent for use in a method for:
a) avoiding and/or reducing cytokine release syndrome;
b) inhibiting cytokine secretion by myeloid cells; and/or
c) killing myeloid cells
in a subject.
